(19) United States
(12) Patent Application Publication
(10) Pub. No.: US 2023/0024832 A1
(43) Pub. Date: Jan. 26, 2023

(54) LEATHER-LIKE MATERIAL COMPRISING MELT-BLENDED COLLAGEN AND THERMOPLASTIC POLYMER

(71) Applicant: Modern Meadow, Inc., Nutley, NJ (US)

(72) Inventors: Shaobo CAI, Oradell, NJ (US); Dale Lee HANDLIN, JR., Clifton, NJ (US); Bryce Michael KRIEGMAN, Jersey City, NJ (US); Nicholas Andrew YARAGHI, Hoboken, NJ (US)

(21) Appl. No.: 17/783,570

(22) PCT Filed: Dec. 9, 2020
(86) PCT No.: PCT/US2020/063915

§ 371 Date: Jun. 8, 2022

Related U.S. Application Data
(60) Provisional application No. 62/992,321, filed on Mar. 20, 2020.
(60) Provisional application No. 62/945,552, filed on Dec. 9, 2019.

(51) Int. Cl. D06N 3/10; D06N 3/00

(52) U.S. Cl. CPC D06N 3/106; D06N 3/0077; D06N 3/0059; D06N 3/0088; D06N 3/0002

(57) ABSTRACT

The present disclosure relates to thermoplastic collagen elastomer compositions comprising collagen, at least one reactive thermoplastic elastomer, and at least one softener, as well as composite materials made from these compositions. Methods of making and using the thermoplastic collagen elastomer composite materials to produce engineered leather are also disclosed.

### FIELD

The present disclosure relates to thermoplastic collagen elastomer compositions comprising collagen, at least one reactive thermoplastic elastomer, and at least one softener. Methods of making and using the thermoplastic collagen elastomer composites to produce engineered leather are also disclosed.

### BACKGROUND

Leather is used in a vast variety of applications, such as in furniture, upholstery, clothing, shoes, luggage, handbags and accessories, and in automotive applications. The global trade value for leather is high, and there is a continuing and increasing demand for leather products. However, there are variety of costs, constraints, and social concerns associated with producing natural leather. Natural leathers are produced from the skins of animals which require raising livestock. This requires enormous amounts of feed, pastureland, water, and fossil fuels. It produces air and waterway pollution, including production of greenhouse gases like methane. It also raises social concerns about the treatment of animals. In recent years, there has also been a fairly well documented decrease in the availability of traditional high quality hides. For at least these reasons, alternative means to meet the demand for leather are desirable.

### SUMMARY

This disclosure provides a thermoplastic collagen elastomer composite material comprising collagen that has been reacted with a reactive functional group of a thermoplastic elastomer.

In some embodiments, the reactive functional group is a maleic anhydride, an epoxy, a silane, or a glycidyl group.

This disclosure also provides a thermoplastic collagen elastomer composite material comprising:
(a) collagen having at least one first reactive functional group and/or a collagen-like protein having at least one first reactive functional group; and
(b) at least one thermoplastic elastomer having at least one second reactive functional group;
   wherein the collagen and/or collagen-like protein and the at least one thermoplastic elastomer are covalently bound together through reaction of the first and second reactive functional groups.

In some embodiments, the collagen in the composite material is recombinant collagen.

In some embodiments, the molecular weight of the collagen and/or collagen-like protein in the composite material is about 10 kDa to about 1000 kDa.

In some embodiments, the first reactive functional group in the composite material is an amino group, a hydroxyl group, or a carboxylic acid group.

In some embodiments, the second reactive functional group in the composite material is a maleic anhydride, an epoxy group, a silane, or a glycidyl group.

In some embodiments, the thermoplastic elastomer in the composite material has an elastic modulus of about 1 MPa to about 20 MPa.

In some embodiments, the thermoplastic elastomer in the composite material is a maleated thermoplastic elastomer or a natural rubber derived product.

In some embodiments, the thermoplastic elastomer in the composite material is selected from the group consisting of a maleated polyethylene, a maleated polypropylene, a maleated styrene-ethylene-butene-styrene block copolymer, a maleated styrene-butadiene-styrene block copolymer, a maleated styrene-ethylene-propylene-styrene block copolymer, a maleated ethylene-propylene rubber, an epoxidized natural rubber, and a methyl methacrylate grafted natural rubber.

In some embodiments, the thermoplastic elastomer in the composite material is selected from the group consisting of polystyrene-b/ock-poly(ethylene-ran-butylene)-block-polystyrene-graft maleic anhydride, polyisoprene-graft-maleic anhydride, poly(propylene-graft-maleic anhydride), maleic anhydride-grafted-ethylene-propylene rubber, poly(ethylene-co-methyl acrylate-co-glycidyl methacrylate), polyethylene-graft-maleic anhydride, and epoxidized natural rubber.

In some embodiments, the composite material further comprises an immiscible unreactive thermoplastic elastomer.

In some embodiments, the immiscible unreactive thermoplastic elastomer in the composite material is selected from the group consisting of a polyurethane block copolymer, a copolyether ester block copolymer, a polyamide block copolymer, a polyether block copolymer, an ethylene vinyl acetate, and a block copolymer having the general formula A-B-A′ or A-B.

In some embodiments, the immiscible unreactive thermoplastic elastomer in the composite material is selected from the group consisting of polystyrene-block-poly(ethylene-ran-butylene)-block-polystyrene, polystyrene-block-poly(ethylene-propylene)-block-styrene block copolymer, polyisoprene, polystyrene-block-polyisoprene-block-polystyrene, polybutadiene, polystyrene-block-polybutadiene-block-polystyrene, styrene-ethylene-butylene-styrene, poly(ethylene-co-vinyl acetate), ethylene-propylene rubber, natural rubber, and poly(ethylene-co-ethyl acrylate).

In some embodiments, the composite material is a film.

This disclosure also provides a method of making a thermoplastic collagen elastomer composite material comprising admixing and heating at a temperature from about 80° C. and about 180° C., a mixture comprising:
(i) collagen and/or a collagen-like protein;
(ii) a secondary component; and
(iii) at least one softener;
   wherein the secondary component comprises at least one immiscible reactive thermoplastic elastomer.

In some embodiments, the at least one collagen in the mixture is recombinant collagen.

In some embodiments, the molecular weight of the collagen and/or collagen-like protein in the mixture is about 10 kDa to about 1000 kDa

In some embodiments, the at least one thermoplastic elastomer in the mixture is an immiscible reactive thermoplastic elastomer.

In some embodiments, the immiscible reactive thermoplastic elastomer in the mixture is a maleated thermoplastic elastomer or a natural rubber derived product.

In some embodiments, the immiscible reactive thermoplastic elastomer in the mixture is selected from the group consisting of a maleated polyethylene, a maleated polypropylene, a maleated styrene-ethylene-butene-styrene block copolymer, a maleated styrene-butadiene-styrene block copolymer, a maleated ethylene-propylene rubber, an epoxidized natural rubber, and a methyl methacrylate grafted natural rubber.

In some embodiments, the immiscible reactive thermoplastic elastomer in the mixture is selected from the group consisting of polystyrene-block-poly(ethylene-ran-butylene)-block-polystyrene-graft maleic anhydride, polyisoprene-graft-maleic anhydride, poly(propylene-graft-maleic anhydride), maleic anhydride-grafted-ethylene-propylene rubber, poly(ethylene-co-methyl acrylate-co-glycidyl methacrylate), polyethylene-graft-maleic anhydride, and epoxidized natural rubber.

In some embodiments, the weight (by mass) of the immiscible reactive thermoplastic elastomer in the mixture is from about 10% to about 1000% of the weight of the collagen.

In some embodiments, the mixture further comprises an immiscible unreactive thermoplastic elastomer.

In some embodiments, the mixture further comprises an immiscible unreactive thermoplastic elastomer is selected from the group consisting of a polyurethane block copolymer, a copolyether ester block copolymer, a polyamide block copolymer, a polyether block copolymer, an ethylene vinyl acetate, and a block copolymer having the general formula A-B-A′ or A-B.

In some embodiments, the immiscible unreactive thermoplastic elastomer in the mixture is selected from the group consisting of polystyrene-block-poly(ethylene-ran-butylene)-block-polystyrene, polystyrene-block-poly(ethylene-propylene)-block-styrene block copolymer, polyisoprene, polystyrene-block-polyisoprene-block-polystyrene, polybutadiene, polystyrene-block-polybutadiene-block-polystyrene, styrene-ethylene-butylene-styrene, poly(ethylene-co-vinyl acetate), ethylene-propylene rubber, natural rubber, and poly(ethylene-co-ethyl acrylate).

In some embodiments, the mixture comprises by weight of the collagen from about 10% to about 1000% of the immiscible unreactive thermoplastic elastomer.

In some embodiments, at least one softener in the mixture is an elastomer softener.

In some embodiments, the elastomer softener in the mixture is selected from the group consisting of a mineral oil, a processing oil, and a vegetable oil.

In some embodiments, the elastomer softener in the mixture is selected from the group consisting of a soybean oil, a linseed oil, a castor oil, a sunflower oil, a rubber seed oil, a palm oil, and a coconut oil.

In some embodiments, the mixture comprises by weight of the collagen about 0.1% to about 200% of at least one elastomer softener.

In some embodiments, at least one softener in the mixture is a collagen softener.

In some embodiments, the collagen softener in the mixture is water or an alcohol.

In some embodiments, the alcohol in the mixture is selected from the group consisting of glycerol, ethylene glycol, diethylene glycol, triethylene glycol, tetraethylene glycol, polyethylene glycol, sorbitol, mannitol, xylitol, 1,4-butanediol, meso-erythritol, and adonitol.

In some embodiments, the mixture comprises by weight of the collagen about 1% to about 100% of at least one collagen softener.

In some embodiments, the admixing is at an agitation rate of from about 20 rpm to about 1000 rpm.

In some embodiments, the admixing is performed over a period of from about 1 minute to about 15 minutes.

In some embodiments, the immiscible reactive thermoplastic elastomer in the mixture has an elastic modulus of about 1 MPa to about 20 MPa.

In some embodiments, the method further comprises hot-pressing the thermoplastic collagen elastomer composite to form a thermoplastic collagen composite film.

In some embodiments, the method further comprises depositing the thermoplastic collagen composite onto a fabric.

This disclosure also provides a thermoplastic collagen elastomer composition suitable for preparing the thermoplastic collagen composite, the composition comprising:
(a) collagen and/or a collagen-like protein;
(b) at least one immiscible reactive thermoplastic elastomer; and
(c) at least one softener.

In some embodiments, the collagen in the composition is a recombinant collagen.

In some embodiments, the molecular weight of the collagen in the composition is about 10 kDa to about 1000 kDa.

In some embodiments, the immiscible reactive thermoplastic elastomer in the composition is a maleated thermoplastic elastomer or a natural rubber derived product.

In some embodiments, the immiscible reactive thermoplastic elastomer in the composition is selected from the group consisting of a maleated polyethylene, a maleated polypropylene, a maleated styrene-ethylene-butene-styrene block copolymer, a maleated styrene-butadiene-styrene block copolymer, a maleated ethylene-propylene rubber, an epoxidized natural rubber, and a methyl methacrylate grafted natural rubber.

In some embodiments, the immiscible reactive thermoplastic elastomer in the composition is selected from the group consisting of polystyrene-block-poly(ethylene-ran-butylene)-block-polystyrene-graft maleic anhydride, polyisoprene-graft-maleic anhydride, poly(propylene-graft-maleic anhydride), maleic anhydride-grafted-ethylene-propylene rubber, poly(ethylene-co-methyl acrylate-co-glycidyl methacrylate), polyethylene-graft-maleic anhydride, and epoxidized natural rubber.

In some embodiments, the composition comprises by weight of the collagen from about 10% to about 1000% of the immiscible reactive thermoplastic elastomer.

In some embodiments, the immiscible reactive thermoplastic elastomer in the composition has an elastic modulus of about 1 MPa to about 20 MPa.

In some embodiments, the composition further comprises an immiscible unreactive thermoplastic elastomer.

In some embodiments, the immiscible unreactive thermoplastic elastomer in the composition is selected from the group consisting of a polyurethane block copolymer, a copolyether ester block copolymer, a polyamide block copolymer, a polyether block copolymer, an ethylene vinyl acetate, and a block copolymer having the general formula A-B-A′ or A-B.

In some embodiments, the immiscible unreactive thermoplastic elastomer in the composition is selected from the group consisting of polystyrene-block-poly(ethylene-ran-butylene)-block-polystyrene, polystyrene-block-poly(ethylene-propylene)-block-styrene block copolymer, polyisoprene, polystyrene-block-polyisoprene-block-polystyrene, polybutadiene, polystyrene-block-polybutadiene-block-polystyrene, styrene-ethylene-butylene-styrene, poly(ethylene-co-vinyl acetate), ethylene-propylene rubber, natural rubber, and poly(ethylene-co-ethyl acrylate).

In some embodiments, the composition comprises by weight of the collagen from about 10% to about 1000% of the immiscible unreactive thermoplastic elastomer.

In some embodiments, at least one softener in the composition is a collagen softener.

In some embodiments, the collagen softener in the composition is water or an alcohol.

In some embodiments, the collagen softener in the composition is selected from the group consisting of glycerol, ethylene glycol, diethylene glycol, triethylene glycol, tetraethylene glycol, polyethylene glycol, sorbitol, mannitol, xylitol, 1,4-butanediol, meso-erythritol, and adonitol.

In some embodiments, the composition comprises by weight of the collagen about 1% to about 100% of at least one collagen softener.

In some embodiments, at least one softener in the composition is an elastomer softener.

In some embodiments, the elastomer softener in the composition is selected from the group consisting of a mineral oil, a processing oil, and a vegetable oil.

In some embodiments, the elastomer softener in the composition is selected from the group consisting of a soybean oil, a linseed oil, a castor oil, a sunflower oil, a rubber seed oil, a palm oil, and a coconut oil.

In some embodiments, the composition comprises by weight of the collagen about 0.1% to about 200% of at least one elastomer softener.

This disclosure also provides an article comprising the composite material described herein.

In some embodiments, the article is selected from the group consisting of footwear, garments, gloves, furniture, vehicle upholstery, overcoats, coats, jackets, shirts, trousers, pants, shorts, swimwear, undergarments, uniforms, emblems, letters, costumes, ties, skirts, dresses, blouses, leggings, gloves, mittens, shoes, shoe components, dress shoes, athletic shoes, running shoes, casual shoes, fashion shoes, boots, sandals, buttons, sandals, hats, masks, headgear, headbands, head wraps, belts, jewelry, gloves, umbrellas, walking sticks, wallets, mobile phones, wearable computer coverings, purses, backpacks, suitcases, handbags, folios, folders, boxes, hunting gear, recreational gear, book bindings, book covers, picture frames, artwork, furnishings, wall coverings, ceiling coverings, flooring, automobile products, boat products, and aircraft products.

### BRIEF DESCRIPTION OF THE DRAWINGS

FIG. 1 is a scatterplot comparing the maximum tensile strength of a film comprising collagen, SEBS-30-MA, and EVA-40 (Formulation 1) to a film comprising SEBS-30-MA and EVA-40 without collagen. As shown in FIG. 1, the film comprising collagen has a higher maximum tensile strength than the film without collagen.

FIG. 2 is a scatterplot comparing the normalized tear strength of a film comprising collagen, SEBS-30-MA, and EVA-40 (Formulation 1) to a film comprising SEBS-30-MA and EVA-40 without collagen. As shown in FIG. 2, the film comprising collagen has a lower normalized tear strength than the film without collagen.

FIG. 3 is a scatterplot comparing the elastic modulus of a film comprising collagen, SEBS-30-MA, and EVA-40 (Formulation 1) to a film comprising SEBS-30-MA and EVA-40 without collagen. As shown in FIG. 3, the film comprising collagen has a higher elastic modulus than the film without collagen.

FIG. 4 is a scatterplot comparing the elongation at failure of a film comprising collagen, SEBS-30-MA, and EVA-40 (Formulation 1) to a film comprising SEBS-30-MA and EVA-40 without collagen. As shown in FIG. 4, the film comprising collagen has a lower elongation at failure than the film without collagen.

FIG. 5 is a line graph comparing the thermal stability of a film comprising collagen, SEBS-30-MA, and EVA-40 (Formulation 1) to a film comprising SEBS-30-MA and EVA-40 without collagen. As shown in FIG. 5, the film comprising polymer and collagen softens but does not melt up to 150° C. The film comprising polymer alone (without collagen) melts around 140° C.

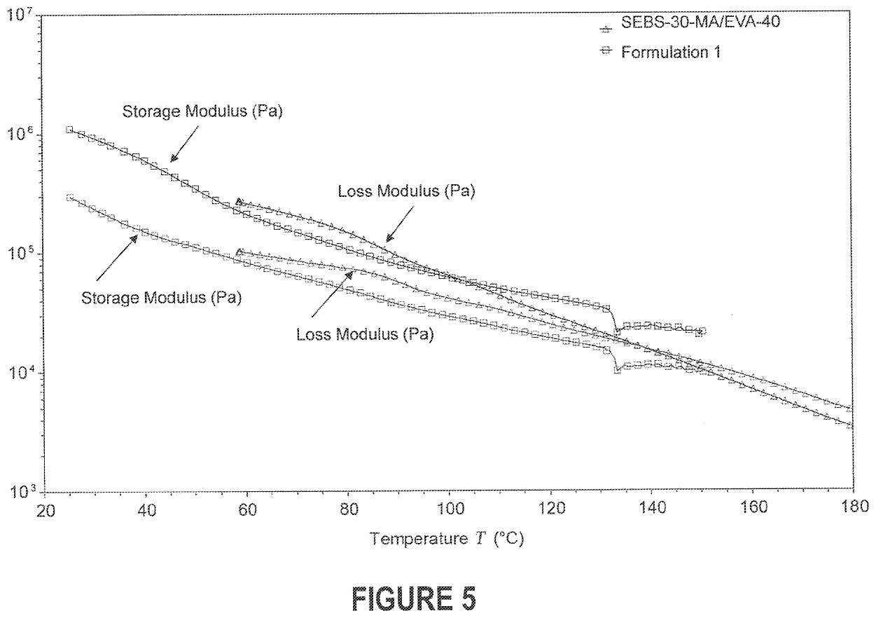

FIG. 6 is a table showing the mechanical properties of four formulations comprising at least one reactive thermoplastic elastomer and collagen.

### DETAILED DESCRIPTION

### Definitions

As used herein, “a,” “an,” and “the” include the plural referents unless the context clearly dictates otherwise. The terms “a” or “an,” as well as the terms “one or more,” and “at least one” can be used interchangeably herein.

As used in the claims, “comprising” is an open-ended transitional phrase. A list of elements following the transitional phrase “comprising” is a non-exclusive list, such that elements in addition to those specifically recited in the list can also be present. As used in the claims, “consisting essentially of” or “composed essentially of” limits the composition of a material to the specified materials and those that do not materially affect the basic and novel characteristic(s) of the material. As used in the claims, “consisting of” or “composed entirely of” limits the composition of a material to the specified materials and excludes any material not specified.

Where a range of numerical values is recited herein, comprising upper and lower values, unless otherwise stated in specific circumstances, the range is intended to include the endpoints thereof, and all integers and fractions within the range. It is not intended that the scope of the claims be limited to the specific values recited when defining a range. Further, when an amount, concentration, or other value or parameter is given as a range, one or more preferred ranges or a list of upper preferable values and lower preferable values, this is to be understood as specifically disclosing all ranges formed from any pair of any upper range limit or preferred value and any lower range limit or preferred value, regardless of whether such pairs are separately disclosed. Finally, when the term “about” is used in describing a value or an end-point of a range, the disclosure should be understood to include the specific value or end-point referred to. Whether or not a numerical value or end-point of a range recites “about,” the numerical value or end-point of a range is intended to include two embodiments: one modified by “about,” and one not modified by “about.”

As used herein, the term “about” refers to a value that is within ±10% of the value stated. For example, about 3 kPa can include any number between 2.7 kPa and 3.3 kPa.

As used herein, the term “thermoplastic elastomer” refers to a polymer that (1) has the ability to be stretched beyond its original length and retract to substantially its original length when released; and (2) softens when exposed to heat and returns to substantially it original condition when cooled to room temperature. In some embodiments, and most typically, the thermoplastic elastomers are not crosslinked and are otherwise void of crosslinking.

As used herein, the term “immiscible” is intended to mean not forming a homogeneous mixture when combined with a collagen at room temperature. The immiscibility of a thermoplastic elastomer with a collagen in a composition can depend on the ratio of thermoplastic elastomer to collagen, the molecular weight of the collagen and the thermoplastic elastomer used in the composition, and the presence of other additional components in the composition that affect the compatibility of the collagen and thermoplastic elastomer. Temperature can also affect the segregation of the thermoplastic elastomer and the collagen in the composition. As used herein, an “immiscible thermoplastic elastomer” refers to a thermoplastic elastomer that cannot be uniformly mixed or blended with collagen to form a single-phase under suitable process conditions.

As used herein, the term “unreactive” is intended to mean chemically unreactive, i.e. inert, to collagen.

As used herein, the term “maleated” refers to a polymer in which maleic anhydride has been grafted onto the polymer backbone.

As used herein, an “unreactive thermoplastic elastomer” refers to a thermoplastic elastomer that does not chemically react with collagen.

As used herein, the term “compatibilizer” refers to a compound which has separate portions in its molecule, wherein one portion is essentially soluble in or has an affinity for collagen and another portion which is essential soluble in or has an affinity for an immiscible thermoplastic elastomer. A compatibilizer lowers the interfacial energy between the components in a composition by having an affinity for both components. Compatibilizers also allow for energy transfer across the phase boundary. Compatibilizers further enhance the ability to disperse immiscible thermoplastic elastomers, however, the extent of the dispersing ability of a particular compatibilizer depends on many different factors. In some embodiments, the compatibilizer can be a block copolymer, graft copolymer, star copolymer, radial copolymer, or an organic or inorganic compound that has an affinity for both components in a composition. In some embodiments, the compatibilizer can be a reactive immiscible thermoplastic elastomer.

As used herein, the term “softener” refers to a substance or material incorporated into another material (usually a plastic or elastomer) to increase its flexibility, workability, or flowability.

As used herein, the term “alkyl” refers to a straight or branched, saturated, aliphatic radical having the number of carbon atoms indicated. In some embodiments, the alkyl is C₁₋₂ alkyl, C₁₋₃ alkyl, C₁₋₄ alkyl, C₁₋₅ alkyl, or C₁₋₆ alkyl. For example, C₁₋₆ alkyl includes, but is not limited to, methyl, ethyl, propyl, isopropyl, butyl, isobutyl, sec-butyl, tert-butyl, pentyl, isopentyl, and hexyl. In some embodiments, the alkyl is octyl, nonyl, decyl, undecyl, dodecyl, tridecyl, tetradecyl, pentadecyl, hexadecyl, heptadecyl, octadecyl, nonadecyl, or icosanyl.

As used herein “collagen” refers to the family of at least 28 distinct naturally occurring collagen types including, but not limited to collagen types I, II, III, IV, V, VI, VII, VIII, IX, X, XI, XII, XIII, XIV, XV, XVI, XVII, XVIII, XIX, and XX. The term collagen as used herein also refers to collagen prepared using recombinant techniques. The term collagen includes collagen, collagen fragments, collagen-like proteins, triple helical collagen, alpha chains, monomers, gelatin, trimers and combinations thereof. Recombinant expression of collagen and collagen-like proteins is known in the art (see, European Patent No. 1232182 and U.S. Pat. Nos. 6,428,978 and 8,188,230, incorporated by reference herein in their entireties) Unless otherwise specified, collagen of any type, whether naturally occurring or prepared using recombinant techniques, can be used in any of the embodiments described herein. That said, in some embodiments, the composite materials described herein can be prepared using Bovine Type I collagen.

As used herein, a collagen described as “bound to” a thermoplastic elastomer means that the collagen is directly bound to a thermoplastic elastomer by a covalent or ionic bond. A collagen described as “bound to” an immiscible reactive thermoplastic elastomer can be bound to the immiscible reactive thermoplastic elastomer by a covalent bond. For example, it is believed that hydroxyl and/or amino groups in collagen can react with anhydride, epoxide, or other reactive functional groups present on the immiscible reactive thermoplastic elastomer to form a covalent bond linking the collagen to the polymer. The nature of the linkage, i.e. the number of atoms separating the collagen from the polymer's backbone and the nature of the covalent bond (i.e. carbon-oxygen, carbon-nitrogen, etc.), will vary depending on the placement and nature of the functional group(s) present on a given immiscible reactive thermoplastic elastomer.

As used herein, the phrase “disposed on” means that a first component (e.g., layer or substrate) is in direct contact with a second component. A first component “disposed on” a second component can be deposited, formed, placed, or otherwise applied directly onto the second component. In other words, if a first component is disposed on a second component, there are no components between the first component and the second component. A surface treatment, such as a surface functionalization treatment, is not considered a component disposed between a first component and a second component.

As used herein, the term “thermoplastic collagen elastomer composite material” refers to the product formed after blending, heating, and cooling a thermoplastic collagen elastomer composition. In some embodiments, the thermoplastic collagen elastomer composite material refers to the product formed after collagen and an immiscible reactive thermoplastic elastomer react (a “covalent collagen polymer composite”).

As used herein the term, “collagen-like protein” refers to truncated proteins derived from human collagen as well as proteins found in non-human cells, such as bacteria of fungi, wherein the proteins have a Gly-Xaa-Yaa repeating amino acid sequence and a length that can be the same or different compared to animal collagen.

The present disclosure provides thermoplastic collagen elastomer compositions, thermoplastic collagen elastomer composite materials, and methods of making thermoplastic collagen elastomer composite materials, that have a look and feel, as well as mechanical properties, similar to natural leather. The thermoplastic collagen elastomer composite materials can have, among other things, haptic properties, aesthetic properties, mechanical/performance properties, manufacturability properties, and/or thermal properties similar to natural leather.

Mechanical/performance properties that can be similar to natural leather include, but are not limited to, tensile strength, tear strength, elongation at break, resistance to abrasion, internal cohesion, water resistance, and the ability to retain color when rubbed (color fastness). Haptic properties that can be similar to natural leather include, but are not limited to, softness, rigidity, coefficient of friction, and compression modulus. Aesthetic properties that can be similar to natural leather include, but are not limited to, dye-ability, embossing, aging, color, color depth, and color patterns. Manufacturing properties that can be similar to natural leather include, but are not limited to, the ability to be stitched, cut, skived, and split. Thermal properties that can be similar to natural leather include, but are not limited to, heat resistance and resistance to stiffening or softening over a significantly wide temperature range, for example 25° C. to 100° C.

### Thermoplastic Collagen Elastomer Compositions

The present disclosure provides thermoplastic collagen elastomer compositions. These compositions are suitable for preparing thermoplastic collagen elastomer composite materials after treatment under suitable conditions described elsewhere herein. For example, and in some embodiments, subjecting a thermoplastic collagen elastomer composition to heat provides a thermoplastic collagen elastomer composite material.

In one embodiment, the present disclosure provides a thermoplastic collagen elastomer composition comprising:
(a) collagen and/or a collagen-like protein;
(b) at least one reactive thermoplastic elastomer; and
(c) at least one softener.

In some embodiments, the thermoplastic collagen elastomer composition further comprises at least one unreactive thermoplastic elastomer.

### Collagen

Collagens are characterized by a repeating triplet of amino acids, -(Gly-X-Y)n-, so that approximately one-third of the amino acid residues in collagen are glycine. X is often proline and Y is often hydroxyproline. Thus, the structure of collagen can consist of three intertwined peptide chains of differing lengths. Different animals can produce different amino acid compositions of the collagen, which can result in different properties (and differences in the resulting leather). Collagen triple helices (also called monomers or tropocollagen) can be produced from alpha-chains of about 1050 amino acids long, so that the triple helix takes the form of a rod of about approximately 300 nm long, with a diameter of approximately 1.5 nm. In the production of extracellular matrix by fibroblast skin cells, triple helix monomers can be synthesized and the monomers can self-assemble into a fibrous form. These triple helices can be held together by electrostatic interactions (including salt bridging), hydrogen bonding, Van der Waals interactions, dipole-dipole forces, polarization forces, hydrophobic interactions, and covalent bonding. Triple helices can be bound together in bundles called fibrils, and fibrils can further assemble to create fibers and fiber bundles. In some embodiments, fibrils can have a characteristic banded appearance due to the staggered overlap of collagen monomers. This banding can be called “D-banding.” The bands are created by the clustering of basic and acidic amino acids, and the pattern is repeated four times in the triple helix (D-period). (See, e.g., Covington, A., Tanning Chemistry: The Science of Leather (2009)) The distance between bands can be approximately 67 nm for Type 1 collagen. These bands can be detected using diffraction Transmission Electron Microscope (TEM), which can be used to assess the degree of fibrillation in collagen. Fibrils and fibers typically branch and interact with each other throughout a layer of skin. D-banding can also be observed using bright field imaging. Variations of the organization or crosslinking of fibrils and fibers can provide strength to a material disclosed herein. In some embodiments, protein is formed, but the entire collagen structure is not triple helical. In certain embodiments, the collagen structure can be about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99% or 100% triple helical.

Regardless of the type of collagen, all are formed and stabilized through a combination of physical and chemical interactions including electrostatic interactions (including salt bridging), hydrogen bonding, Van der Waals interactions, dipole-dipole forces, polarization forces, hydrophobic interactions, and covalent bonding often catalyzed by enzymatic reactions. For Type I collagen fibrils, fibers, and fiber bundles, its complex assembly is achieved in vivo during development and is critical in providing mechanical support to the tissue while allowing for cellular motility and nutrient transport.

Various distinct collagen types have been identified in vertebrates, including bovine, ovine, porcine, chicken, and human collagens. Generally, the collagen types are numbered by Roman numerals, and the chains found in each collagen type are identified by Arabic numerals. Detailed descriptions of structure and biological functions of the various different types of naturally occurring collagens are generally available in the art; see, e.g., Ayad et al. (1998) The Extracellular Matrix Facts Book, Academic Press, San Diego, Calif.; Burgeson, R E., and Nimmi (1992) “Collagen types: Molecular Structure and Tissue Distribution” in Clin. Orthop. 282:250-272; Kielty, C. M. et al. (1993) “The Collagen Family: Structure, Assembly And Organization In The Extracellular Matrix,” Connective Tissue And Its Heritable Disorders, Molecular Genetics, And Medical Aspects, Royce, P. M. and B. Steinmann eds., Wiley-Liss, NY, pp. 103-147; and Prockop, D. J- and K. I. Kivirikko (1995) “Collagens: Molecular Biology, Diseases, and Potentials for Therapy,” Annu. Rev. Biochem., 64:403-434.)

Type I collagen is the major fibrillar collagen of bone and skin, comprising approximately 80-90% of an organism's total collagen. Type I collagen is the major structural macromolecule present in the extracellular matrix of multicellular organisms and comprises approximately 20% of total protein mass. Type I collagen is a heterotrimeric molecule comprising two α1(I) chains and one α2(I) chain, encoded by the COL1A1 and COL1A2 genes, respectively. Other collagen types are less abundant than type I collagen, and exhibit different distribution patterns. For example, type II collagen is the predominant collagen in cartilage and vitreous humor, while type III collagen is found at high levels in blood vessels and to a lesser extent in skin.

Type II collagen is a homotrimeric collagen comprising three identical a1(II) chains encoded by the COL2A1 gene. Purified type II collagen can be prepared from tissues by, methods known in the art, for example, by procedures described in Miller and Rhodes (1982) Methods In Enzymology 82:33-64.

Type III collagen is a major fibrillar collagen found in skin and vascular tissues. Type III collagen is a homotrimeric collagen comprising three identical α1(III) chains encoded by the COL3A1 gene. Methods for purifying type III collagen from tissues can be found in, for example, Byers et al. (1974) Biochemistry 13:5243-5248; and Miller and Rhodes, supra.

Type IV collagen is found in basement membranes in the form of sheets rather than fibrils. Most commonly, type IV collagen contains two α1(IV) chains and one α2(IV) chain. The particular chains comprising type IV collagen are tissue-specific. Type IV collagen can be purified using, for example, the procedures described in Furuto and Miller (1987) Methods in Enzymology, 144:41-61, Academic Press.

Type V collagen is a fibrillar collagen found in, primarily, bones, tendon, cornea, skin, and blood vessels. Type V collagen exists in both homotrimeric and heterotrimeric forms. One form of type V collagen is a heterotrimer of two α1(V) chains and one α2(V) chain. Another form of type V collagen is a heterotrimer of α1(V), α2(V), and α3(V) chains. A further form of type V collagen is a homotrimer of α1(V). Methods for isolating type V collagen from natural sources can be found, for example, in Elstow and Weiss (1983) Collagen Rel. Res. 3:181-193, and Abedin et al. (1982) Biosci. Rep. 2:493-502.

Type VI collagen has a small triple helical region and two large non-collagenous remainder portions. Type VI collagen is a heterotrimer comprising α1(VI), α2(VI), and α3(VI) chains. Type VI collagen is found in many connective tissues. Descriptions of how to purify type VI collagen from natural sources can be found, for example, in Wu et al. (1987) Biochem. J. 248:373-381, and Kielty et al. (1991) J. Cell Sci. 99:797-807.

Type VII collagen is a fibrillar collagen found in particular epithelial tissues. Type VII collagen is a homotrimeric molecule of three al(VII) chains. Descriptions of how to purify type VII collagen from tissue can be found in, for example, Lunstrum et al. (1986) J. Biol. Chem. 261:9042-9048, and Bentz et al. (1983) Proc. Natl. Acad. Sci. USA 80:3168-3172. Type VIII collagen can be found in Descemet's membrane in the cornea. Type VIII collagen is a heterotrimer comprising two α1(VIII) chains and one α2(VIII) chain, although other chain compositions have been reported. Methods for the purification of type VIII collagen from nature can be found, for example, in Benya and Padilla (1986) J. Biol. Chem. 261:4160-4169, and Kapoor et al. (1986) Biochemistry 25:3930-3937.

Type IX collagen is a fibril-associated collagen found in cartilage and vitreous humor. Type IX collagen is a heterotrimeric molecule comprising α1(IX), a2(IX), and a3 (IX) chains. Type IX collagen has been classified as a FACIT (Fibril Associated Collagens with Interrupted Triple Helices) collagen, possessing several triple helical domains separated by non-triple helical domains. Procedures for purifying type IX collagen can be found, for example, in Duance, et al. (1984) Biochem. J. 221:885-889; Ayad et al. (1989) Biochem. J. 262:753-761; and Grant et al. (1988) The Control of Tissue Damage, Glauert, A. M., ed., Elsevier Science Publishers, Amsterdam, pp. 3-28.

Type X collagen is a homotrimeric compound of α1(X) chains. Type X collagen has been isolated from, for example, hypertrophic cartilage found in growth plates. (See, e.g., Apte et al. (1992) Eur JBiochem 206 (1):217-24.)

Type XI collagen can be found in cartilaginous tissues associated with type II and type IX collagens, and in other locations in the body. Type XI collagen is a heterotrimeric molecule comprising α1(XI), α2(XI), and α3(XI) chains. Methods for purifying type XI collagen can be found, for example, in Grant et al., supra.

Type XII collagen is a FACIT collagen found primarily in association with type I collagen. Type XII collagen is a homotrimeric molecule comprising three α1(XII) chains. Methods for purifying type XII collagen and variants thereof can be found, for example, in Dublet et al. (1989) J. Biol. Chem. 264:13150-13156; Lunstrum et al. (1992) J. Biol. Chem. 267:20087-20092; and Watt et al. (1992) J. Biol. Chem. 267:20093-20099.

Type XIII is a non-fibrillar collagen found, for example, in skin, intestine, bone, cartilage, and striated muscle. A detailed description of type XIII collagen can be found, for example, in Juvonen et al. (1992) J. Biol. Chem. 267: 24700-24707.

Type XIV is a FACIT collagen characterized as a homotrimeric molecule comprising α1(XIV) chains. Methods for isolating type XIV collagen can be found, for example, in Aubert-Foucher et al. (1992) J. Biol. Chem. 267:15759-15764,and Watt et al., supra.

Type XV collagen is homologous in structure to type XVIII collagen. Information about the structure and isolation of natural type XV collagen can be found, for example, in Myers et al. (1992) Proc. Natl. Acad. Sci. USA 89:10144-10148; Huebner et al. (1992) Genomics 14:220-224; Kivirikko et al. (1994) J. Biol. Chem. 269:4773-4779; and Muragaki, J. (1994) Biol. Chem. 264:4042-4046.

Type XVI collagen is a fibril-associated collagen, found, for example, in skin, lung fibroblast, and keratinocytes. Information on the structure of type XVI collagen and the gene encoding type XVI collagen can be found, for example, in Pan et al. (1992) Proc. Natl. Acad. Sci. USA 89:6565-6569; and Yamaguchi et al. (1992) J. Biochem. 112:856-863.

Type XVII collagen is a hemidesmosal transmembrane collagen, also known at the bullous pemphigoid antigen. Information on the structure of type XVII collagen and the gene encoding type XVII collagen can be found, for example, in Li et al. (1993) J. Biol. Chem. 268(12):8825-8834; and McGrath et al. (1995) Nat. Genet. 11(1):83-86.

Type XVIII collagen is similar in structure to type XV collagen and can be isolated from the liver. Descriptions of the structures and isolation of type XVIII collagen from natural sources can be found, for example, in Rehn and Pihlajaniemi (1994) Proc. Natl. Acad. Sci USA 91:4234-4238; Oh et al. (1994) Proc. Natl. Acad. Sci USA 91:4229-4233; Rehn et al. (1994) J. Biol. Chem. 269:13924-13935; and Oh et al. (1994) Genomics 19:494-499.

Type XIX collagen is believed to be another member of the FACIT collagen family, and has been found in mRNA isolated from rhabdomyosarcoma cells. Descriptions of the structures and isolation of type XIX collagen can be found, for example, in Inoguchi et al. (1995) J. Biochem. 117:137-146; Yoshioka et al. (1992) Genomics 13:884-886; and Myers et al., J. Biol. Chem. 289:18549-18557 (1994).

Type XX collagen is a newly found member of the FACIT collagenous family, and has been identified in chick cornea. (See, e.g., Gordon et al. (1999) FASEB Journal 13:A1119; and Gordon et al. (1998), IOVS 39:S1128.)

Any type of collagen, truncated collagen, unmodified or post-translationally modified, or amino acid sequence-modified collagen can be used to produce the thermoplastic collagen elastomer composite materials described herein. The degree of incorporation of the collagen molecules can be determined via x-ray diffraction. This characterization will provide d-spacing values which will correspond to different periodic structures present (e.g., 67 nm spacing vs. amorphous). Additional methods for detecting the degree of incorporation of collagen molecules, for example when x-ray diffraction does not provide conclusive results, include Fourier-transform infrared spectroscopy (FTIR), differential scanning calorimetry (DSC), rheology, and mechanical testing. In some embodiments, the collagen can be substantially homogenous collagen, such as only Type I or Type III collagen or can contain mixtures of two or more different kinds of collagens. In embodiments, the collagen is recombinant collagen.

For example, a thermoplastic collagen elastomer composition can contain a single type of collagen molecule, for example 100% bovine Type I collagen or 100% Type III bovine collagen, or can contain a mixture of different kinds of collagen molecules or collagen-like molecules, such as a mixture of bovine Type I and Type III molecules. The collagen mixtures can include amounts of each of the individual collagen components in the range of about 1% to about 99%, including subranges. For example, the amounts of each of the individual collagen components within the collagen mixtures can be about 1%, about 10%, about 20%, about 30%, about 40%, about 50%, about 60%, about 70%, about 80%, about 90%, or about 99%, or within a range having any two of these values as endpoints. For example, in some embodiments, a collagen mixture can contain about 30% Type I collagen and about 70% Type III collagen. Or, in some embodiments, a collagen mixture can contain about 33.3% of Type I collagen, about 33.3% of Type II collagen, and about 33.3% of Type III collagen, where the percentage of collagen is based on the total mass of collagen in the composition or on the molecular percentages of collagen molecules.

In other embodiments, the collagen can be recombinant collagen. In still further embodiments, the collagen can be a mixture of natural and recombinant collagen. In some embodiments, a collagen mixture can contain about 10% natural collagen and about 90% natural collagen, about 20% natural collagen and about 80% recombinant collagen, about 30% natural collagen and about 70% recombinant collagen, about 40% natural collagen and about 60% recombinant collagen, about 50% natural collagen and about 50% recombinant collagen, about 40% natural collagen and about 60% recombinant collagen, about 30% natural collagen and about 70% recombinant collagen, about 20% natural collagen and about 80% recombinant collagen, or about 10% natural collagen and about 90% recombinant collagen, wherein the percentage of collagen is based on the total mass of collagen in the mixture.

In some embodiments, the molecular weight of the collagen is about 10 kDa to about 1000 kDa. In some embodiments, the molecular weight of the collagen is about 10 kDa to about 1000 kDa, about 10 kDa to about 500 kDa, about 10 kDa to about 400 kDa, about 10 kDa to about 300 kDa, about 10 kDa to about 200 kDa, about 10 kDa to about 100 kDa, about 10 kDa to about 50 kDa, about 50 kDa to about 1000 kDa, about 50 kDa to about 500 kDa, about 50 kDa to about 400 kDa, about 50 kDa to about 300 kDa, about 50 kDa to about 200 kDa, about 50 kDa to about 100 kDa, about 100 kDa to about 1000 kDa, about 100 kDa to about 500 kDa, about 100 kDa to about 400 kDa, about 100 kDa to about 300 kDa, about 100 kDa to about 200 kDa, about 200 kDa to about 1000 kDa, about 200 kDa to about 500 kDa, about 200 kDa to about 400 kDa, about 200 kDa to about 300 kDa, about 300 kDa to about 1000 kDa, about 300 kDa to about 500 kDa, about 300 kDa to about 400 kDa, about 400 kDa to about 1000 kDa, about 400 kDa to about 500 kDa, or about 500 kDa to about 1000 kDa.

Collagen with a molecular weight of about 10 kDa to about 100 kDa can be too stiff for use in the formation of a biofabricated material. In some embodiments, where the molecular weight of the collagen is about 10 kDa to about 100 kDa a softener is used with the collagen to allow it to be blended with other materials and/or hot pressed into a film. In some embodiments, where the molecular weight of the collagen is about 20 kDa to about 100 kDa, a softener is used with the collagen to allow it to be blended with other materials and/or hot pressed into a film.

### Thermoplastic Elastomer

In some embodiments, the composition comprises at least one reactive thermoplastic elastomer. In some embodiments, the composition can comprise one to five, one to four, one to three, one to two, two to five, two to four, two to three, three to five, three to four, or four to five thermoplastic elastomers. In some embodiments, the composition can comprise one, two, three, four, or five thermoplastic elastomers. In some embodiments, the composition can comprise one thermoplastic elastomers. In some embodiments, the composition can comprise two thermoplastic elastomers.

Thermoplastic elastomers are a class of copolymers that consist of materials with both thermoplastic and elastomeric properties. There are six main classes of thermoplastic elastomers: (1) styrenic block copolymers; (2) thermoplastic polyolefin elastomers; (3) thermoplastic vulcanizates; (4) thermoplastic polyurethanes; (5) thermoplastic copolyester; and (6) thermoplastic polyamides. Thermoplastic elastomer compositions are versatile because they exhibit beneficial elastomeric properties and can be processed using standard thermoplastic processing equipment. In order to qualify as a thermoplastic elastomer, a material must possess the following characteristics: (1) the ability to be stretched to moderate elongation and, upon the removal of stress, return to something close to its original shape; (2) processable as a melt at elevated temperature; and (3) absence of significant creep.

Due to a wide difference in polarity between collagen and a nonpolar thermoplastic elastomer, collagen typically does not disperse easily in nonpolar thermoplastic elastomers. Instead, collagen tends to agglomerate during mixing with nonpolar thermoplastic elastomers due to the tendency to form strong intermolecular hydrogen bonds with other collagen molecules. As a result of the poor compatibility and dispersability of collagen with nonpolar thermoplastic elastomers, it is difficult to obtain composite materials. It has been surprisingly discovered, however, that the compatibility of collagen with nonpolar thermoplastic elastomers can be improved by introducing reactive groups such as anhydrides and epoxides into the structure of the nonpolar thermoplastic elastomer (“Immiscible Reactive Thermoplastic Elastomers”).

### Immiscible Reactive Thermoplastic Elastomer

In some embodiments, the at least one thermoplastic elastomer is an immiscible reactive thermoplastic elastomer. An immiscible reactive thermoplastic elastomer is chemically reactive with collagen under normal processing conditions. Without wishing to be bound by a particular theory, it is believed that hydroxyl and/or amino groups in collagen react with anhydride groups, epoxide groups, or other reactive functional groups present in the immiscible reactive thermoplastic elastomer such that the collagen is covalently linked to the immiscible reactive thermoplastic elastomer (a covalent collagen polymer composite). Because collagen contains multiple hydroxyl and/or amino groups that can react with multiple functional groups present in the immiscible reactive thermoplastic elastomer, the functional groups in collagen can react with functional groups present on one or more polymeric chains in the immiscible reactive thermoplastic elastomers. That is, the collagen can behave as a cross-liking agent.

In some embodiments, the immiscible reactive thermoplastic elastomer acts as a compatibilizer to facilitate blending collagen and an immiscible unreactive thermoplastic elastomer, as described in detail elsewhere herein. For example, collagen can react with an immiscible reactive thermoplastic elastomer to form a covalent collagen polymer composite, which can be, in certain embodiments, simultaneously melt blended with an immiscible unreactive thermoplastic elastomer. Absent forming the covalent collagen polymer composite, collagen would not be soluble or miscible in the immiscible unreactive thermoplastic elastomer.

In some embodiments, the composition comprises at least one immiscible reactive thermoplastic elastomer. In some embodiments, the composition comprises one to five, one to four, one to three, one to two, two to five, two to four, two to three, three to five, three to four, or four to five immiscible reactive thermoplastic elastomers. In some embodiments, the composition comprises one, two, three, four, or five immiscible reactive thermoplastic elastomers. In some embodiments, the composition comprises one immiscible reactive thermoplastic elastomer.

The elastic modulus (or Young's modulus) is the measure of a material's resistance to elastic deformation under tensile loading. When a material is loaded under tension, it undergoes an initial linear relationship between stress (force per unit area) and strain (change in length divided by initial length). The elastic modulus is the slope of the stress-strain curve within the linear elastic region, measured by dividing the stress over strain in this region. The elastic modulus can be measured using the method disclosed in ISO 527.

In embodiments where the immiscible reactive thermoplastic elastomer is the only elastomer in the composition, the immiscible reactive thermoplastic elastomer can have an elastic modulus of about 1 MPa to about 20 MPa. In embodiments where the immiscible reactive thermoplastic elastomer is the only elastomer in the composition, the immiscible reactive thermoplastic elastomer can have an elastic modulus of about 1 MPa to about 20 MPa, about 1 MPa to about 16 MPa, about 1 MPa to about 12 MPa, about 1 MPa to about 10 MPa, about 1 MPa to about 8 MPa, about 1 MPa to about 6 MPa, about 1 MPa to about 4 MPa, about 4 MPa to about 20 MPa, about 4 MPa to about 16 MPa, about 4 MPa to about 12 MPa, about 4 MPa to about 10 MPa, about 4 MPa to about 8 MPa, about 4 MPa to about 6 MPa, about 6 MPa to about 20 MPa, about 6 MPa to about 16 MPa, about 6 MPa to about 12 MPa, about 6 MPa to about 10 MPa, about 6 MPa to about 8 MPa, about 8 MPa to about 20 MPa, about 8 MPa to about 16 MPa, about 8 MPa to about 12 MPa, about 8 MPa to about 10 MPa, about 10 MPa to about 20 MPa, about 10 MPa to about 16 MPa, about 10 MPa to about 12 MPa, about 12 MPa to about 20 MPa, about 12 MPa to about 16 MPa, or about 16 MPa to about 20 MPa.

In embodiments where the immiscible reactive thermoplastic elastomer is admixed with at least one other thermoplastic elastomer in the composition, the blend of thermoplastic elastomers can have an elastic modulus of about 2 MPa to about 10 MPa. In embodiments where the immiscible reactive thermoplastic elastomer is admixed with at least one other thermoplastic elastomer in the composition, the blend of thermoplastic elastomers can have an elastic modulus of about 2 MPa to about 10 MPa, about 2 MPa to about 8 MPa, about 2 MPa to about 6 MPa, about 2 MPa to about 4 MPa, about 4 MPa to about 10 MPa, about 4 MPa to about 8 MPa, about 4 MPa to about 6 MPa, about 6 MPa to about 10 MPa, about 6 MPa to about 8 MPa, or about 8 MPa to about 10 MPa.

In some embodiments, the immiscible reactive thermoplastic elastomer can be a maleated thermoplastic elastomer. In some embodiments, the maleated thermoplastic elastomer can be a maleated polyethylene, a maleated polypropylene, a maleated styrenic block copolymer such as maleated styrene-ethylene-butene-styrene block copolymer, maleated styrene-butadiene-styrene block copolymer, or maleated styrene-ethylene-propylene-styrene block copolymer, or a maleated ethylene-propylene rubber.

In some embodiments, the immiscible reactive thermoplastic elastomer can be a natural rubber derived product such as an epoxidized natural rubber or a methyl methacrylate grafted natural rubber.

In some embodiments, the immiscible reactive thermoplastic elastomer can be polystyrene-block-poly(ethylene-ran-butylene)-block-polystyrene-graft maleic anhydride, polyisoprene-graft-maleic anhydride, poly(propylene-graft-maleic anhydride), maleic anhydride-grafted-ethylene-propylene rubber, poly(ethylene-co-methyl acrylate-co-glycidyl methacrylate), polyethylene-graft-maleic anhydride, or epoxidized natural rubber.

In some embodiments, the composition can comprise by weight of the collagen about 10% to about 1000% of the at least one immiscible reactive thermoplastic elastomer. In some embodiments, the composition can comprise by weight of the collagen about 10% to about 1000%, about 10% to about 500%, about 10% to about 200%, about 10% to about 100%, about 10% to about 75%, about 10% to about 50%, about 50% to about 1000%, about 50% to about 500%, about 50% to about 200%, about 50% to about 100%, about 50% to about 75%, about 75% to about 1000%, about 75% to about 500%, about 75% to about 200%, about 75% to about 100%, about 100% to about 1000%, about 100% to about 500%, about 100% to about 200%, about 200% to about 1000%, about 200% to about 500%, or about 500% to about 1000% of the at least one immiscible reactive thermoplastic elastomer. In some embodiments, the composition can comprise by weight of the collagen about 75% to about 1000% of the at least one immiscible reactive thermoplastic elastomer.

In some embodiments, the composition can further comprise an immiscible unreactive thermoplastic elastomer. An immiscible unreactive thermoplastic elastomer is a thermoplastic elastomer that is not reactive, i.e. inert, to collagen.

In some embodiments, the composition can further comprise at least one immiscible unreactive thermoplastic elastomer. In some embodiments, the composition can further comprise one to five, one to four, one to three, one to two, two to five, two to four, two to three, three to five, three to four, or four to five immiscible unreactive thermoplastic elastomers. In some embodiments, the composition can further comprise one, two, three, four, or five immiscible unreactive thermoplastic elastomers. In some embodiments, the composition can further comprise one immiscible unreactive thermoplastic elastomer.

In some embodiments, the immiscible unreactive thermoplastic polymer can be made from block copolymers such as polyurethanes, copolyether esters, polyamides, polyether block copolymers, ethylene vinyl acetates, or block copolymers having the general formula A-B-A′ or A-B. In some embodiments, block copolymers having the general formula A-B-A′ or A-B include copoly(styrene/ethylene-butylene), poly(styrene-(ethylene-propylene)-styrene), styrene-poly(ethylene-butylene)-styrene, and poly(styrenelethyle-butylene)-styrene).

In some embodiments, the immiscible unreactive thermoplastic elastomer can be polystyrene-block-poly(ethylene-ran-butylene)-block-polystyrene, polystyrene-block-poly(ethylene-propylene)-block-styrene-block copolymer, polyisoprene, polystyrene-block-polyisoprene-block-polystyrene, polybutadiene, polystyrene-block-polybutadiene-block-polystyrene, styrene-ethylene-butylene-styrene, poly(ethylene-co-vinyl acetate), ethylenepropylene rubber, natural rubber, or poly(ethylene-co-ethyl acrylate).

In some embodiments, the composition can comprise by weight of the collagen about 10% to about 1000% of the at least one immiscible unreactive thermoplastic elastomer. In some embodiments, the composition comprises by weight of the collagen about 10% to about 1000%, about 10% to about 500%, about 10% to about 200%, about 10% to about 100%, about 10% to about 75%, about 10% to about 50%, about 50% to about 1000%, about 50% to about 500%, about 50% to about 200%, about 50% to about 100%, about 50% to about 75%, about 75% to about 1000%, about 75% to about 500%, about 75% to about 200%, about 75% to about 100%, about 100% to about 1000%, about 100% to about 500%, about 100% to about 200%, about 200% to about 1000%, about 200% to about 500%, or about 500% to about 1000% of the at least one immiscible unreactive thermoplastic elastomer. In some embodiments, the composition can comprise by weight of the collagen about 75% to about 1000% of the at least one immiscible unreactive thermoplastic elastomer.

### Softener

Softeners can be incorporated into another material (usually a plastic or elastomer) to increase its flexibility, workability, or flowability. In some embodiments, a softener can be incorporated into a collagen to increase its flexibility, workability, or flowability. In some embodiments, a softener can be incorporated into a thermoplastic elastomer to increase its flexibility, workability, or flowability. In some embodiments, a softener can be incorporated into both a collagen and a thermoplastic elastomer. In some embodiments, the softener incorporated into a collagen is the same as the softener incorporated into the thermoplastic elastomer. In some embodiments, the softener incorporated into a collagen is different from the softener incorporated into the thermoplastic elastomer. A change in the type and level of a softener will affect the properties of the final flexible product. The selection for a specific polymer or elastomer is normally based on the compatibility between components; the amount required for plasticization; processing characteristics; desired thermal, electrical, and mechanical properties of the end product; permanence; resistance to water, chemicals, and solar radiation; toxicity; and cost.

In some embodiments, the composition can comprise at least one softener. In some embodiments, the composition can comprise one to five, one to four, one to three, one to two, two to five, two to four, two to three, three to five, three to four, or four to five softeners. In some embodiments, the composition can comprise one, two, three, four, or five softeners. In some embodiments, the composition can comprise one softener. In some embodiments, the composition can comprise two softeners.

In some embodiments, the softener can be a collagen softener, discussed in detail below. In some embodiments, the softener can be an elastomer softener, also described in detail below.

In some embodiments, the composition can comprise at least one collagen softener. In some embodiments, the composition can comprise at least one elastomer softener. In some embodiments, the compositions can comprise at least one collagen softener and at least one elastomer softener.

### Collagen Softener

In some embodiments, the composition can comprise at least one collagen softener. In some embodiments, the composition comprises one to five, one to four, one to three, one to two, two to five, two to four, two to three, three to five, three to four, or four to five collagen softeners. In some embodiments, the composition can comprise one, two, three, four, or five collagen softeners. In some embodiments, the composition can comprise one collagen softener.

In some embodiments, the collagen softener can be water or an alcohol.

In some embodiments, the collagen softener can be an alcohol. In some embodiments, the collagen softener can be a glycol such as glycerol, ethylene glycol, diethylene glycol, triethylene glycol, tetraethylene glycol, polyethylene glycol, sorbitol, mannitol, xylitol, 1,4-butanediol, meso-erythritol, or adonitol.

In some embodiments, the collagen softener can be polyethylene glycol (PEG) with a molecular weight (Mw) ranging from about 300 to about 20,000. In some embodiments, the collagen softener can be a PEG with a molecular weight (Mw) of 300, 400, 600, 800, 1500, 4000, 10,000, or 20,000. In some embodiments, the collagen softener can be PEG 300 or PEG 400.

In some embodiments, the composition can comprise by weight of the collagen about 1% to about 100% of at least one collagen softener. In some embodiments, the composition can comprise by weight of the collagen about 1% to about 100%, about 1% to about 80%, about 1% to about 60%, about 1% to about 40%, about 1% to about 20%, about 1% to about 10%, about 10% to about 100%, about 10% to about 80%, about 10% to about 60%, about 10% to about 40%, about 10% to about 20%, about 20% to about 100%, about 20% to about 80%, about 20% to about 60%, about 20% to about 40%, about 40% to about 100%, about 40% to about 80%, about 40% to about 60%, about 60% to about 100%, about 60% to about 80%, or about 80% to about 100% of at least one collagen softener. In some embodiments, the composition can comprise by weight of the collagen about 80% to about 100% of at least one collagen softener.

### Elastomer Softener

In some embodiments, the composition can comprise at least one elastomer softener. In some embodiments, the composition can comprise one to five, one to four, one to three, one to two, two to five, two to four, two to three, three to five, three to four, or four to five elastomer softeners. In some embodiments, the composition can comprise one, two, three, four, or five elastomer softeners. In some embodiments, the composition can comprise one elastomer softener.

In some embodiments, the elastomer softener can be a mineral oil, a processing oil, or a vegetable oil.

In some embodiments, the elastomer softener can be a processing oil. In some embodiments, the processing oil can be a paraffinic oil, a napthenic oil, an aromatic oil, or a natural oil. In some embodiments, the elastomer softener can be a paraffinic hydrocarbon oil. In some embodiments, the elastomer softener can be a paraffinic hydrocarbon oil having a molecular weight from about 200 to about 1,000.

In some embodiments, the elastomer softener can be a mineral oil.

In some embodiments, the elastomer softener can be a vegetable oil. In some embodiments, the vegetable oil can be a soybean oil, a linseed oil, a castor oil, a sunflower oil, a rubber seed oil, a palm oil, or a coconut oil.

In some embodiments, the composition can comprise by weight of the collagen about 0.1% to about 200% of at least one elastomer softener. In some embodiments, the composition comprises by weight of the collagen about 0.1% to about 200%, about 0.1% to about 100%, about 0.1% to about 60%, about 0.1% to about 40%, about 0.1% to about 20%, about 0.1% to about 10%, about 10% to about 200%, about 10% to about 100%, about 10% to about 60%, about 10% to about 40%, about 10% to about 20%, about 20% to about 200%, about 20% to about 100%, about 20% to about 60%, about 20% to about 40%, about 40% to about 200%, about 40% to about 100%, about 40% to about 60%, about 60% to about 200%, about 60% to about 100%, or about 100% to about 200% of at least one elastomer softener. In some embodiments, the composition can comprise by weight of the collagen about 80% to about 100% of the at least one elastomer softener.

### Methods of Making a Thermoplastic Elastomer Composite

The majority of plastic products are prepared by so-called “hot compounding” techniques, where the ingredients in the composition are combined under heat and shearing forces that bring about a state of molten plastic (fluxing) which is shaped into the desired product, cooled, and allowed to develop ultimate properties of strength and integrity. Hot compounding methods include, but are not limited to, calendering, extrusion, injection, and compression molding.

The present disclosure provides a method of making a thermoplastic collagen elastomer composite material comprising:
(a) admixing:
   (i) collagen and/or collagen-like protein;
   (ii) at least one reactive thermoplastic elastomer;
   (iii) at least one softener; and
(b) heating (a) at a temperature from about 80° C. to about 180° C.

The present disclosure also provides a method of making a thermoplastic collagen elastomer composite material comprising:
(a) admixing:
   (i) collagen and/or collagen-like protein;
   (ii) at least two thermoplastic elastomers, wherein at least one thermoplastic elastomer is a reactive immiscible thermoplastic elastomer and at least one thermoplastic elastomer is an unreactive immiscible thermoplastic elastomer; and
   (iii) at least one softener; and
(b) heating (a) at a temperature from about 80° C. to about 180° C.

In some embodiments, the admixing can be performed at a temperature ranging from about 80° C. to about 180° C., about 80° C. to about 150° C., about 80° C. to about 125° C., about 80° C. to about 100° C., about 100° C. to about 180° C., about 100° C. to about 150° C., about 100° C. to about 125° C., about 125° C. to about 180° C., about 100° C. to about 150° C., or about 150° C. to about 180° C. In some embodiments, the admixing can be performed at a temperature ranging from about 80° C. to about 125° C. In some embodiments, the admixing can be performed at a temperature of about 110° C.

In some embodiments, the admixing can be performed with agitation. In certain embodiments, the admixing can take place at an agitation rate of from about 20 rpm to about 1000 rpm, about 20 rpm to about 500 rpm, about 20 rpm to about 250 rpm, about 20 rpm to about 200 rpm, about 20 rpm to about 100 rpm, about 100 rpm to about 1000 rpm, about 100 rpm to about 500 rpm, about 100 rpm to about 250 rpm, about 100 rpm to about 200 rpm, about 200 rpm to about 1000 rpm, about 200 rpm to about 500 rpm, about 200 rpm to about 250 rpm, about 250 rpm to about 1000 rpm, about 250 rpm to about 500 rpm, or about 500 rpm to about 1000 rpm. In some embodiments, the admixing can take place at an agitation rate of from about 100 rpm to about 200 rpm. In some embodiments, the admixing is in a batch mixer.

In some embodiments, the admixing can be performed over a period of from about 1 minute to about 15 minutes, about 1 minute to about 10 minutes, about 1 minute to about 5 minutes, about 5 minutes to about 15 minutes, about 5 minutes to about 10 minutes, or about 10 minutes to about 15 minutes. In some embodiments, the admixing can be performed over a period of about 10 minutes.

### Thermoplastic Collagen Elastomer Composite Materials

The present disclosure provides a thermoplastic collagen elastomer composite material. In typical embodiments, the thermoplastic collagen elastomer composite material is a covalent collagen polymer composite, the covalent collagen polymer composite material comprising collagen covalently linked to a block copolymer through at least one of an amide, ester, or amino bond.

In some embodiments, the present disclosure provides a thermoplastic elastomer composite material comprising:
(a) collagen and/or collagen-like protein having at least one first reactive functional group; and
(b) at least one thermoplastic elastomer having at least one second reactive functional group;
wherein the collagen and the at least one thermoplastic elastomer are covalently bound together through reaction of the first and second reactive functional groups.

The present disclosure also provides a thermoplastic collagen elastomer composite material comprising:
(a) collagen and/or collagen-like protein having at least one first reactive functional group;
(b) at least one thermoplastic elastomer having at least one second reactive functional group, wherein the at least one thermoplastic elastomer having at least one second reactive functional group is a reactive immiscible thermoplastic elastomer; and wherein the collagen and the at least one thermoplastic elastomer are covalently bound together through reaction of the first and second reactive functional groups.

The present disclosure also provides a thermoplastic collagen elastomer composite material comprising collagen that has been reacted with a reactive thermoplastic elastomer having a functional group.

In some embodiments, the functional group on the reactive thermoplastic elastomer can be selected from the group consisting of a maleic anhydride, an epoxy, a silane, and a glycidyl group.

In some embodiments, the reactive immiscible thermoplastic elastomer can have randomly located functional groups, i.e. a reactive immiscible thermoplastic elastomer with randomly located functional groups. In some embodiments, the reactive immiscible thermoplastic elastomer with randomly located functional groups can be a randomly maleated polymer, a randomly epoxidized polymer, a randomly silanated polymer, or a randomly glycidated polymer.

In some embodiments, the reactive immiscible thermoplastic elastomer with randomly located functional groups can be a randomly maleated polymer. In some embodiments, the randomly maleated polymer can be a randomly maleated polyethylene, a randomly maleated propylene, a randomly maleated ethylene-propylene copolymer (randomly maleated EPR or randomly maleated ethylene-polypropylene rubber), a randomly maleated ethylene-propylene-diene monomer terpolymer (randomly maleated EPDM), a randomly maleated block copolymer such as a styrenic block copolymer, or an acrylic block copolymer. In some embodiments, the randomly maleated polymer is randomly maleated EPR, randomly maleated EPDM, or a randomly maleated styrenic block copolymers such as randomly maleated poly(styrene-block-hydrogenated butadiene-block-styrene) (SEBS) or maleated poly(styrene-block-hydrogenated isoprene-styrene) (SEPS).

In some embodiments, the reactive immiscible thermoplastic elastomer with randomly located functional groups can be a randomly epoxidized polymer. In some embodiments, the randomly epoxidized polymer can be a randomly epoxidized diene containing polymer. In some embodiments, the randomly epoxidized polymer can be a polymer containing isoprene monomer units such as randomly epoxidized natural rubber (ENR), a randomly epoxidized isoprene containing block copolymer such as poly(styrene-block-isoprene) or poly(stryrene-block isoprene-blockstyrene), a randomly epoxidized ethylene-propylene-diene monomer (epoxidized EPDM), or randomly epoxidized polyisobutylene.

In some embodiments, the randomly epoxidized polymer can present its epoxide groups through one or more randomly located glycidyl groups, i.e.

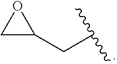 [SMILES: *CC1CO1]

In some embodiments, the reactive immiscible thermoplastic elastomer with randomly located glycidyl groups can be a copolymer of glycidyl methacrylate or a polymer grafted with glycidyl methacrylate.

In some embodiments, the reactive immiscible thermoplastic elastomer with randomly located functional groups can be a randomly silanated polymer. In some embodiments, the randomly silanated polymer can be a randomly silane-functionalized ethylene-propylene-diene monomer (silane-functionalized EPDM), a randomly silanated cellulosic polymer, a randomly silanated polyvinyl alcohol or a partially hydrolyzed polyvinyl acetate, a randomly silane-functionalized polydimethyl siloxane, or a randomly silanated adhesive polymer.

In some embodiments the reactive immiscible thermoplastic elastomer can have groups placed in blocks, as coupling agents, or on the ends. Examples of such polymers include, but are not limited to, block copolymers containing a glycidyl methacrylate block. Polymers coupled with silane coupling agents where functionality is still remaining on the silane coupling agents include styrene-butadiene rubbers and styreninc block copolymers.

In some embodiments, the thermoplastic collagen elastomer composite can be hot-pressed into a film. In some embodiments, the film can have a thickness ranging of from about 0.5 mm to about 50 mm, including subranges. In some embodiments, the film can have a thickness of about 0.5 mm, about 1 mm, about 2 mm, about 3 mm, about 4 mm, about 5 mm, about 10 mm, about 15 mm, about 20 mm, about 25 mm, about 30 mm, about 35 mm, about 40 mm, about 45 mm, or about 50 mm, or within a range having any two of these values as endpoints, inclusive of the endpoints.

In some embodiments, the thermoplastic collagen elastomer composite can be disposed onto a fabric. In some embodiments, the fabric can be made from one or more natural fibers, for example fibers made from cotton, linen, silk, wool, kenaf, flax, cashmere, angora, bamboo, bast, hemp, soya, seacell, milk or milk proteins, spider silk, chitosan, mycelium, cellulose including bacterial cellulose, or wood. In some embodiments, the fabric can be made from one or more synthetic fibers, for example fibers made from polyesters, nylons, aromatic polyamides, polyolefin fibers such as polyethylene, polypropylene, rayon, lyocell, viscose, antimicrobial yarn (A.M.Y.), Sorbtek, nylon, elastomers such as LYCRA, spandex, or ELASTANE, polyester-polyurethane copolymers, aramids, carbon including carbon fibers and fullerenes, glass, silicon, minerals, metals or metal alloys including those containing iron, steel, lead, gold, silver, platinum, copper, zinc, and titanium, or mixtures thereof.

Typically, the thermoplastic collagen elastomer composite materials described herein have physical and mechanical properties similar to those of natural leather. For example, the thermoplastic collagen elastomer composite materials can have similar thickness, tear strength, tensile strength, flexibility, and softness values as those of natural leather.

In some embodiments, the thermoplastic collagen elastomer composite material described herein can have a tear strength that is at least about 1% greater than that of a natural leather of the same thickness. For example, the thermoplastic collagen elastomer composite material can have a tear strength that is about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 100%, about 150%, or about 200% greater than that of natural leather of the same thickness. In some embodiments, the thermoplastic collagen elastomer composite material can have a tear strength in the range of about 20 N to about 500 N, including subranges. For example, the tear strength of the thermoplastic collagen elastomer composite material can be about 20 N, about 30 N, about 40 N, about 50 N, about 60 N, about 70 N, about 80 N, about 90 N, about 100 N, about 125 N, about 150 N, about 175 N, about 200 N, about 225 N, about 250 N, about 275 N, about 300 N, about 325 N, about 350 N, about 375 N, about 400 N, about 425 N, about 450 N, about 475 N, or about 500 N, or within a range having any two of these values as endpoints, inclusive of the endpoints. The tear strength can be normalized. In some embodiments, the tear strength can be normalized by dividing the tear force (N) by the material thickness (nm) to yield a normalized tear strength (N/nm). In some embodiments, the normalized tear strength of the thermoplastic collagen elastomer composite material can be about 2 N/mm to about 30 N/mm. For example, the normalized tear strength of the thermoplastic collagen elastomer composite can be about 2 N/mm to about 30 N/mm, about 2 N/mm to about 20 N/mm, about 2 N/mm to about 10 N/mm, about 2 N/mm to about 5 N/mm, about 5 N/mm to about 30 N/mm, about 5 N/mm to about 20 N/mm, about 5 N/mm to about 10 N/mm, about 10 N/mm to about 30 N/mm, about 10 N/mm to about 20 N/mm, or about 20 N/mm to about 30 N/mm.

Tensile strength, or ultimate tensile strength (UTS), measures the capacity of a material to withstand laods in tension without failing. Tensile strength, or ultimate tensile strength, is defined as the maximum tensile stress a material can withstand without failing. Unless specified otherwise, a tensile strength value disclosed herein is measured according the method provided by ASTM D 412. In some embodiments, the thermoplastic collagen elastomer composite material described herein can have a tensile strength in the range of about 1 kPa (kilopascal) to about 100 MPa (megapascals), including subranges. For example, the thermoplastic collagen elastomer composite material can have a tensile strength of about 1 kPa, about 50 kPa, about 100 kPa, about 200 kPa, about 300 kPa, about 400 kPa, about 500 kPa, about 600 kPa, about 700 kPa, about 800 kPa, about 900 kPa, about 1 MPa, about 5 MPa, about 10 MPa, about 20 MPa, about 30 MPa, about 40 MPa, about 50 MPa, about 60 MPa, about 70 MPa, about 80 MPa, about 90 MPa, or about 100 MPa, or within a range having any two of these values as endpoints, inclusive of the endpoints. In some embodiments, the tensile strength of the thermoplastic collagen elastomer composite material can be about 1 MPa to about 10 MPa.

Ultimate elongation, or strain, of a material can be determined by measuring its elongation at failure when a tensile force is applied, for example using the equation:

$$\frac{Δ L}{L} ,$$

where ΔL is the change in length of the material after the tensile force is applied, and L is the original length of the material. Elongation can also be measured according to the method provided by ASTM D 412. In some embodiments, thermoplastic collagen elastomer composite material described herein can have an elongation in the range of about 1% to about 30%, including subranges. For example, the thermoplastic collagen elastomer composite material can have an elongation of about 1%, about 5%, about 10%, about 15%, about 20%, about 25%, or about 30%, or within a range having any two of these values as endpoints, inclusive of the endpoints. In some embodiments, the thermoplastic collagen elastomer composite material can have an elongation greater than 30%. Unless specified otherwise, an elongation value disclosed herein is measured by ASTM D 412. In some embodiments, the elongation at failure of the thermoplastic collagen elastomer composite material can be about 100% to about 600%. For example, the elongation at failure of the thermoplastic collagen elastomer composite material can be about 100% to about 600%, about 100% to about 500%, about 100% to about 400%, about 100% to about 300%, about 100% to about 200%, about 200% to about 600%, about 200% to about 500%, about 200% to about 400%, about 200% to about 300%, about 300% to about 600%, about 300% to about 500%, about 300% to about 400%, about 400% to about 600%, about 400% to about 500%, or about 500% to about 600%.

The elastic modulus (or Young's modulus) is the measure of the stiffness of a material when it undergoes a tensile strain. When a material is pulled on, it undergoes a linear relationship between stress (force normalized per area) and strain (% elongation). The elastic modulus is measured by dividing the stress over strain in this region (the amount of stress needed to elongate by a certain strain). The elastic modulus can be measured using the method disclosed in ISO 527. In some embodiments, the elastic modulus of the thermoplastic collagen elastomer composite material can be about 1 MPa to about 20 MPa. For example, the elastic modulus of the thermoplastic collagen elastomer composite material can be about 1 MPa to about 20 MPa, about 1 MPa to about 15 MPa, about 1 MPa to about 10 MPa, about 1 MPa to about 5 MPa, about 5 MPa to about 20 MPa, about 5 MPa to about 15 MPa, about 5 MPa to about 10 MPa, about 10 MPa to about 20 MPa, about 10 MPa to about 15 MPa, or about 15 MPa to about 20 MPa.

In some embodiments, the thermoplastic collagen elastomer composite material described herein can be subjected to the same, or similar finishing treatments as those used to treat natural leather. The treatment process for natural leather typically has three steps: preparation of the hide, tanning, retanning, fat-liquoring, and finishing. Tanning can be performed in any number of well-understood ways, including by contacting the thermoplastic collagen elastomer composite material with a vegetable tanning agent, blocked isocyanate compounds, chromium compound, aldehyde, syntan, natural resin, tanning natural oil, or modified oil. Blocked isocyanate compounds can include X-tan. Vegetable tannins can include pyrogallol- or pyrocatechin-based tannins, such as valonea, mimosa, ten, tara, oak, pinewood, sumach, quebracho, and chestnut tannins. Chromium tanning agents can include chromium salts such as chromium sulfate. Aldehyde tanning agents can include glutaraldehyde and oxazolidine compounds. Syntans can include aromatic polymers, polyacrylates, polymethacrylates, copolymers of maleic anhydride and styrene, condensation products of formaldehyde with melamine or dicyandiamide, lignins, and natural flours.

To tan a composite material, the material's pH can be adjusted, for example lowered to a pH in the range of about 2.5 to about 3.0 in the presence of 10% salts (for example sodium chloride, sodium sulfate, or sodium salts), to allow for penetration of the tanning agent. Following penetration, the pH of the composite material can be adjusted again, for example raised to a pH in the range of about 3.5 to about 4.0, to fix the tanning agent. In some embodiments, a composite material can be soaked in a bath including 2 wt. % (based on the weight of collagen infused into the composite material) of chromium (III) sulfate and the pH can be adjusted as necessary for penetration and fixation. For example, for a 10 gram composite material with 10% mass of collagen, 0.02 gram of chrome chromium (III) sulfate powder can be dissolved in enough water to cover the composite material in a container (the amount of water will depend on container dimensions). The composite material can then be added to the container and the container can be agitated, for example on an orbital shaker at 50 rpm. The agitation can be performed at a pH of about 2.8 to about 3.2 and for a time sufficient to allow penetration of the chromium (III) sulfate into the composite material. After penetration, the pH of the bath can be increased and fixation of the chromium (III) sulfate can be performed at a pH between about 3.8 and about 4.2. The duration of the fixation step can be selected to achieve a desired color for the composite material.

In some embodiments, after tanning, the thermoplastic collagen elastomer composite material can be retanned. Retanning refers to post-tanning treatments. Such treatments can include tanning a second time, wetting, sammying, dehydrating, neutralization, adding a coloring agent such as a dye, fat liquoring, fixation of unbound chemicals, setting, conditioning, softening, and/or buffing.

In some embodiments, a coloring agent can be incorporated into a collagen-infused composite material. In some embodiments, the coloring agent can be incorporated into the collagen before reaction with a reactive thermoplastic elastomer. In some embodiments, the coloring agent reacts with the collagen before the collagen reacts with the reactive thermoplastic elastomer.

In some embodiments, the coloring agent can be a dye. In some embodiments, the dye can include one or more chromophores that contain pendant reactive groups capable of forming covalent bonds. These dyes can achieve high wash fastness and a wide range of brilliant shades. Exemplary dyes, include but are not limited to, sulphatoethylsulphone (Remazol), vinylsulphone, and acrylamido dyes. In some embodiments, the dye can be an anionic dye. Exemplary anionic dyes include, but are not limited to, azo, stilbene, phthalocyanine, and dioxazine.

In some embodiments, about 0.01 wt. % to about 7.5 wt. % dye, based on collagen weight, can be used. In some embodiments, the weight percent of dye, based on collagen weight, can be about 0.01 wt. % to about 7.5 wt. %, about 0.01 wt. % to about 5 wt. %, about 0.01 wt. % to about 3 wt. %, about 0.01 wt. % to about 1 wt. %, about 0.01 wt. % to about 0.5 wt. %, about 0.01 wt. % to about 0.1 wt. %, about 0.1 wt. % to about 7.5 wt. %, about 0.1 wt. % to about 5 wt. %, about 0.1 wt. % to about 3 wt. %, about 0.1 wt. % to about 1 wt. %, about 0.1 wt. % to about 0.5 wt. %, about 0.5 wt. % to about 7.5 wt. %, about 0.5 wt. % to about 5 wt. %, about 0.5 wt. % to about 3 wt. %, about 0.5 wt. % to about 1 wt. %, about 1 wt. % to about 7.5 wt. %, about 1 wt. % to about 5 wt. %, about 1 wt. % to about 3 wt. %, about 3 wt. % to about 7.5 wt. %, about 3 wt. % to about 5 wt. %, or about 5 wt. % to about 7.5 wt. %.

In some embodiments, lubricants used during fat liquoring include fats, biological, mineral or synthetic oils, cod oil, sulfonated oil, polymers, organofunctional siloxanes, or other hydrophobic compounds or agents used for fat liquoring conventional leather, or mixtures thereof. Other lubricants can include surfactants, anionic surfactants, cationic surfactants, cationic polymeric surfactants, anionic polymeric surfactants, amphiphilic polymers, fatty acids, modified fatty acids, nonionic hydrophilic polymers, nonionic hydrophobic polymers, poly acrylic acids, poly methacrylic, acrylics, natural rubbers, synthetic rubbers, resins, amphiphilic anionic polymer and copolymers, amphiphilic cationic polymer and copolymers and mixtures thereof as well as emulsions or suspensions of these in water, alcohol, ketones, and other solvents. Lubricants can be incorporated in any amount that facilitates movement of the collagen fibrils, or that confers leather-like properties such as flexibility, decrease in brittleness, durability, or water resistance. In some embodiments, the amount of lubricant applied to a thermoplastic collagen elastomer composite material can be in the range of about 0.1 wt. % to about 60 wt. % of the thermoplastic collagen elastomer composite material. For example, the amount of lubricant applied can be about 0.1 wt. %, about 1 wt. %, about 5 wt. %, about 10 wt. %, about 15 wt. %, about 20 wt. %, about 25 wt. %, about 30 wt. %, about 35 wt. %, about 40 wt. %, about 45 wt. %, about 50 wt. %, about 55 wt. %, or about 60 wt. %, or within a range having any two of these values as endpoints, inclusive of the endpoints.

In some embodiments, during dehydration, water can be removed by filtration, evaporation, freeze-drying, solvent exchange, vacuum-drying, convection-drying, heating, irradiating or microwaving, or by other known methods for removing water.

A tanned thermoplastic collagen elastomer composite material can be mechanically or chemically finished. For example, mechanical finishing can include polishing the composite material to yield a shiny surface; ironing and plating the composite material to achieve a flat, smooth surface; embossing the composite material to create a three-dimensional print or pattern on the material's surface; or tumbling the composite material to provide a more evident grain and smooth surface. Chemical finishing can involve the application of a film, a natural or synthetic coating, or other treatment. Chemical treatments can be applied, for example, by spraying, curtain coating, roller coating, or reverse transfer coating.

The thermoplastic collagen elastomer composite material described herein can be used as a replacement for natural leather in a variety of applications. For example, the thermoplastic collagen elastomer composite material can be used in footwear, garments, gloves, furniture, vehicle upholstery, and other good and products, such as overcoats, coats, jackets, shirts, trousers, pants, shorts, swimwear, undergarments, uniforms, emblems or letters, costumes, ties, skirts, dresses, blouses, leggings, gloves, mittens, shoes, shoe components such as sole, quarter, tongue, cuff, welt, and counter, dress shoes, athletic shoes, running shoes, casual shoes, athletic, running or casual shoe components such as toe cap, toe box, outsole, midsole, upper, laces, eyelets, collar, lining, Achilles notch, heel, and counter, fashion or women's shoes and their shoe components such as upper, outer sole, toe spring, toe box, decoration, vamp, lining, sock, insole, platform, counter, and heel or high heel, boots, sandals, buttons, sandals, hats, masks, headgear, headbands, head wraps, and belts; jewelry such as bracelets, watch bands, and necklaces; gloves, umbrellas, walking sticks, wallets, mobile phone or wearable computer coverings, purses, backpacks, suitcases, handbags, folios, folders, boxes, and other personal objects; athletic, sports, hunting or recreational gear such as harnesses, bridles, reins, bits, leashes, mitts, tennis rackets, golf clubs, polo, hockey, or lacrosse gear, chessboards and game boards, medicine balls, kick balls, baseballs, and other kinds of balls, and toys; book bindings, book covers, picture frames or artwork; furniture and home, office or other interior or exterior furnishings including chairs, sofas, doors, seats, ottomans, room dividers, coasters, mouse pads, desk blotters, or other pads, tables, beds, floor, wall or ceiling coverings, flooring, automobile, boat, aircraft and other vehicular products including seats, headrests, upholstery, paneling, steering wheel, joystick or control coverings and other wraps or coverings.

The embodiments discussed herein will be further clarified in the following examples. It should be understood that these examples are not limiting to the embodiments described above.

### EXAMPLES

### Example 1

### Melt Compounded Collagen and Reactive Immiscible Thermoplastic Elastomer

15 g of recombinant collagen was dissolved in a ˜10 wt % solution of gelatin in water with 15 g of glycerol with stirring and heating at 50-70° C. After 1-2 hours the mixture was cast into a dish, cooled, dried overnight at room temperature (20-25° C.), and dried in an oven at 50-70° C. to form a gelatin/glycerol film. The gelatin/glycerol film was blended with 15 g of polystyrene-block-poly(ethylene-ran-butylene)-block-polystyrene-graft maleic anhydride and 15 g of mineral oil in an ATR (Advanced Torque Rheometer) Plasti-Corder batch mixer (C.W. BRABENDER Instruments, Inc., Hackensack, N.J.). The materials were mixed at 150 rpm at 110° C. for 10 minutes.

The blended materials were then hot-pressed into a stand-alone film or onto a fabric to form leather-like materials. The washed materials can be used directly or after further washing, tanning, and fat-liquoring to modify the properties and haptics.

### Example 2

### Melt Compounded Collagen, Reactive Immiscible Thermoplastic Elastomer, and Immiscible Thermoplastic Elastomer

15 g of recombinant collagen was dissolved in a ˜10 wt % solution of gelatin in water with 15 g of glycerol with stirring and heating at 50-70° C. After 1-2 hours the mixture was cast into a dish, cooled, dried overnight at room temperature (20-25° C.), and dried in an oven at 50-70° C. to form a gelatin/glycerol film. The gelatin/glycerol film was blended with 15 g of polystyrene-block-poly(ethylene-ran-butylene)-block-polystyrene, 5 g of polystyrene-block-poly(ethylene-ran-butylene)-block-polystyrene-graft maleic anhydride, and 15 g of mineral oil in an ATR (Advanced Torque Rheometer) Plasti-Corder batch mixer (C.W. BRABENDER Instruments, Inc., Hackensack, N.J.). The materials were mixed at 150 rpm at 110° C. for 10 minutes. The blended materials were then hot-pressed into a stand-alone film or onto a fabric to form leather-like materials. The washed materials can be used directly or after further washing, tanning, and fat-liquoring to modify the properties and haptics.

### Example 3

### Melt Compounded Collagen, Reactive Immiscible Thermoplastic Elastomer, and Immiscible Thermoplastic Elastomer (Formulation 1)

20 g of recombinant collagen was dissolved in a ˜10 wt % solution of gelatin in water with 3 g of glycerol with stirring and heating at 50-70° C. After 1-2 hours the mixture was cast into a dish, cooled, dried overnight at room temperature (20-25° C.), and dried in an oven at 50-70° C. to form a gelatin/glycerol film. The gelatin/glycerol film was blended with 15 g of polystyrene-block-polyhydrogenated (butadiene)-block-polystyrene-graft maleic anhydride (SEBS-30-MA, Sigma Aldrich, St. Louis, Mo.), 3 g of mineral oil, and 12 g of poly(ethylene-co-vinyl acetate, Sigma Aldrich, St. Louis, MO) (EVA-40) in an ATR (Advanced Torque Rheometer) Plasti-Corder batch mixer (C.W. BRABENDER Instruments, Inc., Hackensack, N.J.). The materials were mixed at 150 rpm at 110° C. for 10 minutes.

The blended materials were then hot-pressed into a stand-alone film.

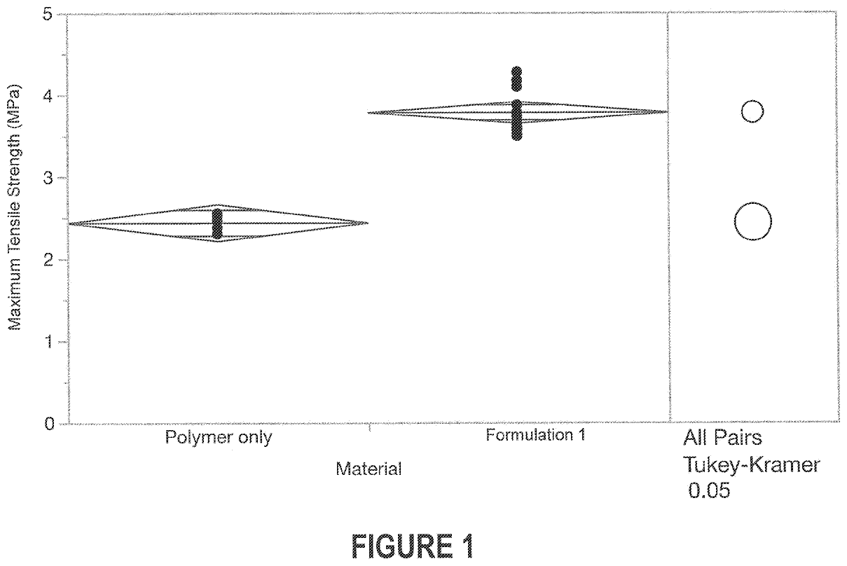

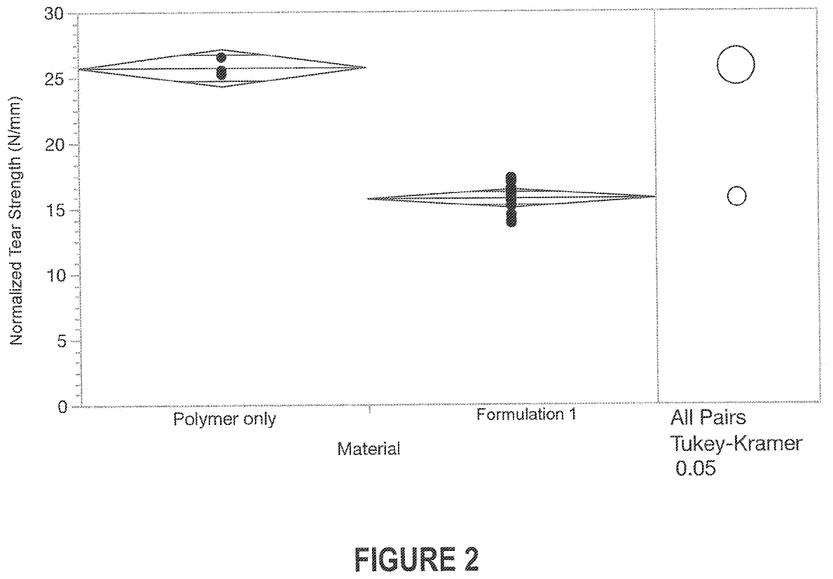

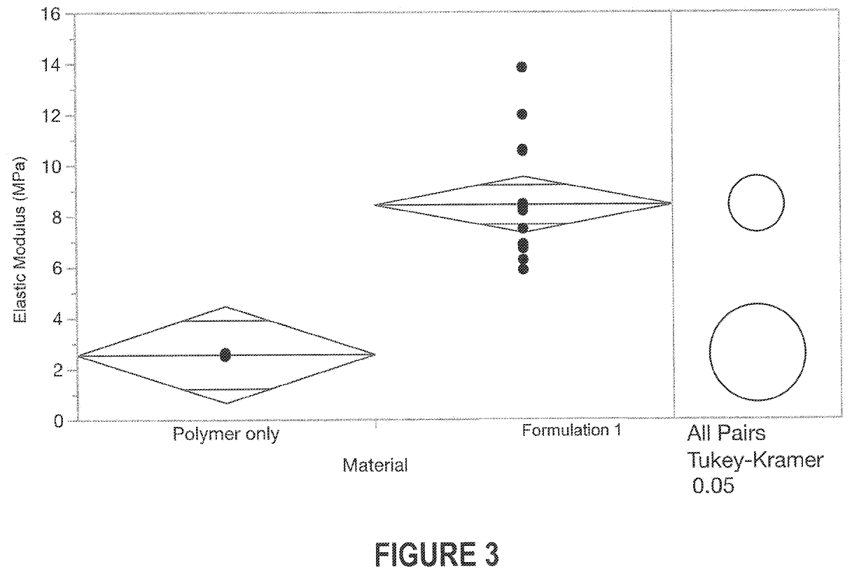

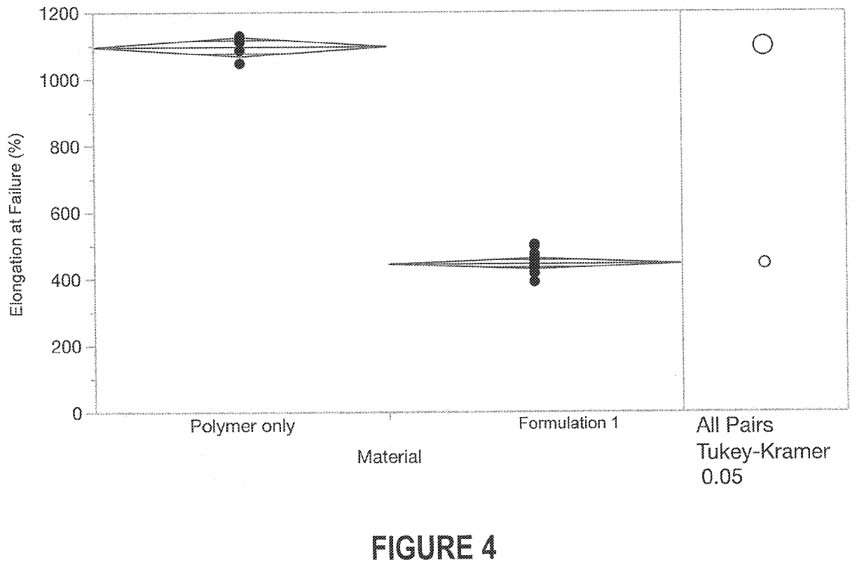

Mechanical properties of the film of Formulation 1 were measured and were compared to a film prepared without collagen. As shown in FIGS. 1-4, compared to a film prepared with polymer alone, a film prepared with Formulation 1 comprising collagen showed higher tensile strength, lower tear strength, higher elastic modulus, and lower elongation at failure. Thus, the addition of collagen to the polymer altered the mechanical properties of the resultant film.

### Example 4

### Melt Compounded Collagen and Reactive Immiscible Thermoplastic Elastomer (Formulation 2)

40 g of recombinant collagen was dissolved in a ˜10 wt % solution of gelatin in water with 10 g of glycerol with stirring and heating at 50-70° C. After 1-2 hours the mixture was cast into a dish, cooled, dried overnight at room temperature (20-25° C.), and dried in an oven at 50-70° C. to form a gelatin/glycerol film. The gelatin/glycerol film was blended with 30 g of poly(ethylene-co-methyl acrylate-co-glycidyl methacrylate) (PEM, Sigma Aldrich, St. Louis, Mo.) in an ATR (Advanced Torque Rheometer) Plasti-Corder batch mixer (C.W. BRABENDER Instruments, Inc., Hackensack, N.J.). The materials were mixed at 150 rpm at 110° C. for 10 minutes.

The blended materials were then hot-pressed into a stand-alone film.

### Example 5

### Melt Compounded Collagen, Reactive Immiscible Thermoplastic Elastomers, and Immiscible Thermoplastic Elastomer (Formulation 3)

20 g of recombinant collagen was dissolved in a ˜10 wt % solution of gelatin in water with 3 g of glycerol with stirring and heating at 50-70° C. After 1-2 hours the mixture was cast into a dish, cooled, dried overnight at room temperature (20-25° C.), and dried in an oven at 50-70° C. to form a gelatin/glycerol film. The gelatin/glycerol film was blended with 2 g of SEBS-30-MA, 8 g of styrene-ethylene-butylene-styrene (SEBS-13-MD, Sigma Aldrich, St. Louis, Mo.), 10 g of ethylene propylene rubber (Bio-EPR, Arlanxeo, Orange, Tex.), and 5 g of mineral oil in an ATR (Advanced Torque Rheometer) Plasti-Corder batch mixer (C.W. BRABENDER Instruments, Inc., Hackensack, N.J.). The materials were mixed at 150 rpm at 110° C. for 10 minutes.

The blended materials were then hot-pressed into a stand-alone film.

### Example 6

### Melt Compounded Collagen and Reactive Immiscible Thermoplastic Elastomer (Formulation 4)

18 g of recombinant collagen was dissolved in a ˜10 wt % solution of gelatin in water with 3 g of glycerol with stirring and heating at 50-70° C. After 1-2 hours the mixture was cast into a dish, cooled, dried overnight at room temperature (20-25° C.), and dried in an oven at 50-70° C. to form a gelatin/glycerol film. The gelatin/glycerol film was blended with 25 g epoxidized natural rubber-50 (ENR-50, MMGuthrie, Phuket, Thailand) in an ATR (Advanced Torque Rheometer) Plasti-Corder batch mixer (C.W. BRABENDER Instruments, Inc., Hackensack, N.J.). The materials were mixed at 150 rpm at 110° C. for 10 minutes.

The blended materials were then hot-pressed into a stand-alone film.

### Example 7

### Mechanical Properties of Formulations 1-4

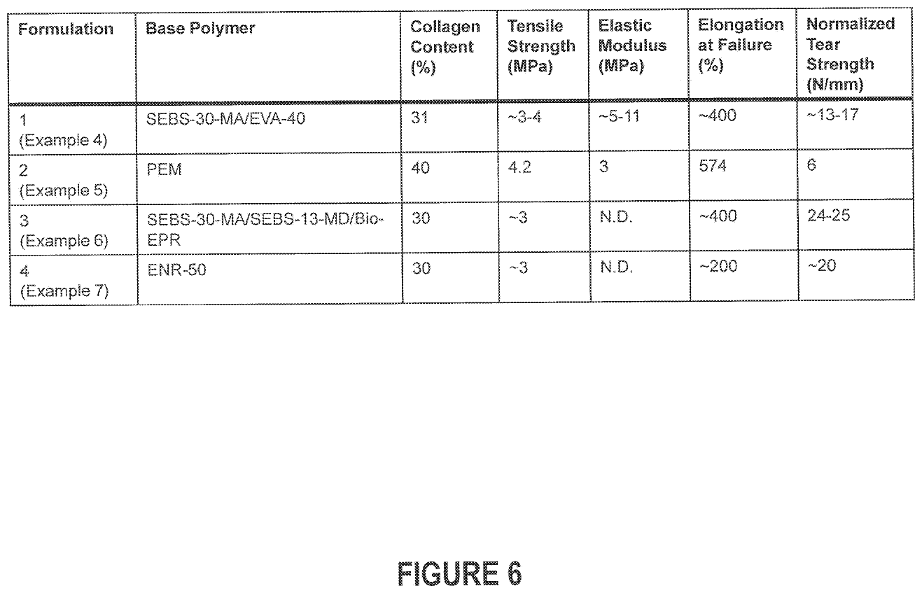

Mechanical properties of the formulations of EXAMPLES 3-6 were measured and are shown in FIG. 6.

While various embodiments have been described herein, they have been presented by way of example, and not limitation. It should be apparent that adaptations and modifications are intended to be within the meaning and range of equivalents of the disclosed embodiments, based on the teaching and guidance presented herein. It therefore will be apparent to one skilled in the art that various changes in form and detail can be made to the embodiments disclosed herein without departing from the spirit and scope of the present disclosure. The elements of the embodiments presented herein are not necessarily mutually exclusive, but can be interchanged to meet various situations as would be appreciated by one of skill in the art.

Embodiments of the present disclosure are described in detail herein with reference to embodiments thereof as illustrated in the accompanying drawings, in which like reference numerals are used to indicate identical or functionally similar elements. References to “one embodiment,” “an embodiment,” “some embodiments,” “in certain embodiments,” etc., indicate that the embodiment described can include a particular feature, structure, or characteristic, but every embodiment can not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The examples are illustrative, but not limiting, of the present disclosure. Other suitable modifications and adaptations of the variety of conditions and parameters normally encountered in the field, and which would be apparent to those skilled in the art, are within the spirit and scope of the disclosure.

It is to be understood that the phraseology or terminology used herein is for the purpose of description and not of limitation. The breadth and scope of the present disclosure should not be limited by any of the above-described exemplary embodiments, but should be defined in accordance with the following claims and their equivalents.

What is claimed is:

1. A thermoplastic collagen elastomer composite material comprising collagen that has been reacted with a reactive thermoplastic elastomer comprising a functional group.

2. The composite material of claim 1, wherein the functional group is selected from the group consisting of a maleic anhydride, an epoxide, a silane, or a glycidyl group.

3. A thermoplastic collagen elastomer composite material comprising:
(a) collagen and/or collagen-like protein having at least one first reactive functional group;
(b) at least one thermoplastic elastomer having at least one second reactive functional group;
wherein the collagen and/or collagen-like protein and the at least one immiscible reactive thermoplastic elastomer are covalently bound together through reaction of the first and second reactive functional groups.

4. The composite material of claim 3, wherein the collagen is recombinant collagen.

5. The composite material of claim 4, wherein the molecular weight of the collagen and/or collagen-like protein is about 10 kDa to about 1000 kDa.

6. The composite material of any one of claims 3-5, wherein the first reactive functional group is an amino group, a hydroxyl group, or a carboxylic acid group.

7. The composite material of any one of claims 3-6, wherein the second reactive functional group is a maleic anhydride, an epoxy group, a silane, or a glycidyl group.

8. The composite material of any one of claims 3-7, wherein the thermoplastic elastomer has an elastic modulus of about 1 MPa to about 20 MPa.

9. The composite material of any one of claims 3-8, wherein the thermoplastic elastomer is a maleated thermoplastic elastomer or a natural rubber derived product.

10. The composite material of any one of claims 3-9, wherein the thermoplastic elastomer is selected from the group consisting of a maleated polyethylene, a maleated polypropylene, a maleated styrene-ethylene-butene-styrene block copolymer, a maleated styrene-butadiene-styrene block copolymer, a maleated styrene-ethylene-propylene-styrene block copolymer, a maleated ethylene-propylene rubber, an epoxidized natural rubber, and a methyl methacrylate grafted natural rubber.

11. The composite material of any one of claims 3-10, wherein the thermoplastic elastomer is selected from the group consisting of polystyrene-block-poly(ethylene-ran-butylene)-block-polystyrene-graft maleic anhydride, polyisoprene-graft-maleic anhydride, poly(propylene-graft-maleic anhydride), maleic anhydride-grafted-ethylene-propylene rubber, poly(ethylene-co-methyl acrylate-co-glycidyl methacrylate), polyethylene-graft-maleic anhydride, and epoxidized natural rubber.

12. The composite material of any one of claims 3-11, further comprising an immiscible unreactive thermoplastic elastomer.

13. The composite material of claim 12, wherein the immiscible unreactive thermoplastic elastomer is selected from the group consisting of a polyurethane block copolymer, a copolyether ester block copolymer, a polyamide block copolymer, a polyether block copolymer, an ethylene vinyl acetate, and a block copolymer having the general formula A-B-A′ or A-B.

14. The composite material of claim 12 or 13, wherein the immiscible unreactive thermoplastic elastomer is selected from the group consisting of polystyrene-block-poly(ethylene-ran-butylene)-block-polystyrene, polystyrene-block-poly(ethylene-propylene)-block-styrene block copolymer, polyisoprene, polystyrene-block-polyisoprene-block-polystyrene, polybutadiene, polystyrene-block-polybutadiene-block-polystyrene, styrene-ethylene-butylene-styrene, poly(ethylene-co-vinyl acetate), ethylene-propylene rubber, natural rubber, and poly(ethylene-co-ethyl acrylate).

15. The composite material of any one of claims 8-14, wherein the composite material is a film.

16. A method of making a thermoplastic collagen elastomer composite material comprising admixing and heating at a temperature from about 80° C. and about 180° C. a mixture comprising:
(a) collagen and/or collagen-like protein; p1 (b) at least one immiscible reactive thermoplastic elastomer; and
(c) at least one softener.

17. The method of claim 16, wherein the collagen is recombinant collagen.

18. The method of claim 16 or 17, wherein the molecular weight of the collagen and/or collagen-like protein is about 10 kDa to about 1000 kDa

19. The method of any one of claims 16-18, wherein the at least one thermoplastic elastomer is an immiscible reactive thermoplastic elastomer.

20. The method of any one of claims 16-19, wherein the weight (by mass) of the immiscible reactive thermoplastic elastomer is from about 10% to about 1000% of the weight of the collagen.

21. The method of any one of claims 16-20, further comprising an immiscible unreactive thermoplastic elastomer.

22. The method of claim 21, comprising by weight of the collagen from about 10% to about 1000% of the immiscible unreactive thermoplastic elastomer.

23. The method of any one of claims 16-22, wherein at least one softener is an elastomer softener.

24. The method of claim 23, wherein the elastomer softener is selected from the group consisting of a mineral oil, a processing oil, and a vegetable oil.

25. The method of any one of claims 16-24, wherein at least one softener is a collagen softener.

26. The method of claim 25, wherein the collagen softener is water or an alcohol.

27. The method of any one of claims 16-26, further comprising hot-pressing the thermoplastic collagen elastomer composite to form a thermoplastic collagen composite film.

28. The method of any one of claims 16-27, further comprising depositing the thermoplastic collagen composite onto a fabric.

29. An article comprising the composite material of any one of claims 1-15.

30. The article of claim 29, wherein the article is selected from the group consisting of footwear, garments, gloves, furniture, vehicle upholstery, overcoats, coats, jackets, shirts, trousers, pants, shorts, swimwear, undergarments, uniforms, emblems, letters, costumes, ties, skirts, dresses, blouses, leggings, gloves, mittens, shoes, shoe components, dress shoes, athletic shoes, running shoes, casual shoes, fashion shoes, boots, sandals, buttons, sandals, hats, masks, headgear, headbands, head wraps, belts, jewelry, gloves, umbrellas, walking sticks, wallets, mobile phones, wearable computer coverings, purses, backpacks, suitcases, handbags, folios, folders, boxes, hunting gear, recreational gear, book bindings, book covers, picture frames, artwork, furnishings, wall coverings, ceiling coverings, flooring, automobile products, boat products, and aircraft products.
